(19) United States
(12) Patent Application Publication
(10) Pub. No.: US 2025/0137076 A1
(43) Pub. Date: May 1, 2025

(54) SET OF SNP MARKERS RELATED TO HEAT TOLERANCE OF PISUM SATIVUM DEVELOPED BASED ON SNAPSHOT TECHNOLOGY, AND USE THEREOF

(71) Applicants: Shandong Academy of Agricultural Sciences, Jinan (CN); Institute of Crop Science, Chinese Academy of Agricultural Sciences, Beijing (CN)

(72) Inventors: Dong Wang, Jinan (CN); Tao Yang, Jinan (CN); Hanfeng Ding, Jinan (CN); Xuxiao Zong, Jinan (CN); Nana Li, Jinan (CN); Rong Liu, Jinan (CN); Qian Tian, Jinan (CN)

(21) Appl. No.: 18/927,753

(22) Filed: Oct. 25, 2024

Related U.S. Application Data
(63) Continuation of application No. PCT/CN2023/073450, filed on Jan. 21, 2023.

(30) Foreign Application Priority Data
Apr. 25, 2022 (CN) 202210439663.8

(51) Int. Cl. C12Q 1/6895

(52) U.S. Cl. CPC C12Q 1/6895; C12Q 2600/13; C12Q 2600/156

(57) ABSTRACT

The present invention discloses a set of SNP markers related to heat tolerance of Pisum sativum developed based on a SnaPshot technology, and use thereof. In the present invention, 2,358 accessions of Pisum sativum germplasms from all over the world are subjected to a heat tolerance screening experiment by employing a manner of sowing by stages, and a reasonable Pisum sativum heat-tolerant classifying standard is established through identification of yield-related traits, so that a Pisum sativum germplasm population including heat-tolerant and heat-sensitive germplasms is obtained; and the population is subjected to genetic diversity and population genetic structure analysis through a set of SNP markers (20 markers) related to heat tolerance of Pisum sativum developed based on an SNaPshot technology, so as to provide theoretical and practical basis for the future study of heat-tolerant genetic mechanism and heat-tolerant breeding of Pisum sativum.

### FIELD OF TECHNOLOGY

The present invention relates to a set of SNP markers related to heat tolerance of Pisum sativum developed based on an SNaPshot technology, and use thereof, and belongs to the technical field of plant genetics.

### BACKGROUND

The trend of global warming in the future is becoming more and more obvious. By 2018, the global temperature had increased by 1.5° C. compared with that before industrialization, and it is expected that the average ground surface temperature will increase by 1.5-2.0° C. by the end of this century. Heat stress is one of the main abiotic stresses that lead to plant yield loss. In a range from 1° C. to 5° C., the yield will be lost by 10% per 1° C. of increase in the temperature, and it occurs even in heat-tolerant plants such as sorghum bicolor. Cold-season edible beans, including Pisum sativum, Cicer arietinum L., Lens culinaris Medikus etc., will suffer from yield loss due to various environmental stresses, and the effect of heat stress on them is especially serious during flowering and seed development.

China is a big country in production and consumption of Pisum sativum As a cold-season crop, Pisum sativum is particularly sensitive to high temperature in the field, and the high temperature can cause abortion of flowers, fruits and seeds and decrease of seed size in Pisum sativum Under high temperature conditions, a heat-tolerant variety shows more genital segments generated per plant, less loss of pods per plant and less loss of seeds per pod, while a heat-sensitive variety shows the opposite. Therefore, the seed yield per plant is an important index to measure whether Pisum sativum is heat-tolerant. Heat-tolerant varieties obtained by screening are important gene sources for future genetic improvement of Pisum sativum

Understanding the genetic diversity and population genetic relationship among Pisum sativum germplasms is of great importance for the genetic improvement research of Pisum sativum and the selection of suitable parents in breeding of it. Many markers have been utilized by predecessors to evaluate the genetic diversity of Pisum sativum germplasm, among which simple sequence repeats (SSRs) are widely used because of their rich polymorphism, high stability and low cost. Single nucleotide polymorphisms (SNPs) are the third generation of molecular markers after restriction fragment length polymorphisms (RFLPs) and simple sequence repeats (SSRs). The SNPs have the advantages of a diallelic property, abundant quantity, wide distribution, low mutation rate, and being capable of realizing automatic high-throughput detection.

The SNPs can be typed by various detection means, such as PCR-single strand conformation polymorphism (PCR-SSCP), allele-specific PCR (AS-PCR) and cleaved amplified polymorphic sequence (CAPS), as well as next generation sequencing (NGS), kompetitive allele-specific PCR (KASP), minisequencing and the like which can achieve medium and high throughput SNP typing. An SNaPshot technology is a technology of multiplex analysis for SNPs, which can achieve medium-throughput typing of SNPs. It has the characteristics of high sensitivity, good repeatability, no need for additional equipment, and the like. It has been widely used in forensic identification, SNP detection of human genes and the like research fields, and has been reported in plant genetics research fields such as SNP typing and marker development, molecular marker-assisted breeding and genetic diversity analysis, and the like. Up to now, the SNP marker is seldom applied in aspects of evaluation of genetic diversity of Pisum sativum and study of population genetic structure, with only sporadic reports of it, while the research of Pisum sativum in the genetic aspect by using the SNaPshot technology has been rarely reported.

Currently, 3,837 accessions of Pisum sativum germplasms collected from more than 40 countries and regions are deposited in National Crop Germplasm Bank of China, but the identification data of their phenotypic traits are not perfect. Only partial traits of them have been preliminarily identified, such as identification of cold tolerance, but they have never been screened and identified for heat tolerance on a large scale. Zong Xuxiao e tal. have utilized an SSR marker to evaluate the genetic diversity and population genetic structure of 2,017 accessions of Pisum sativum germplasms collected all over the world and deposited in the National Crop Germplasm Bank of China, but have never conducted analysis of the genetic diversity and population structure of Pisum sativum germplasms subjected to heat stress treatment.

### SUMMARY

In view of the aforementioned problems, in the present invention, 2,358 accessions of Pisum sativum germplasms from all over the world, which are deposited in the National Crop Gene Bank of the Institute of Crop Sciences, Chinese Academy of Agricultural Sciences, are subjected to a heat tolerance screening experiment by employing a manner of sowing by stages, and a reasonable Pisum sativum heat-tolerant classifying standard is established through identification of yield-related traits, so that a Pisum sativum germplasm population including heat-tolerant and heat-sensitive germplasms is obtained; and the population is subjected to genetic diversity and population genetic structure analysis through a set of SNP markers (20 markers) related to heat tolerance of Pisum sativum developed based on an SNaPshot technology, so as to provide theoretical and practical basis for the future study of heat-tolerant genetic mechanism and heat-tolerant breeding of Pisum sativum

The technical solution of the present invention is: a method for conducting heat tolerance screening of Pisum sativum germplasms, including:
sowing Pisum sativum germplasms to be screened in three sowing dates: (1) normal sowing (NS); (2) sowing in a first stage of late sowing (LS1) (15 days later than the date of normal sowing); and (3) sowing in a second stage of late sowing (LS2) (30 days later than the date of normal sowing);
utilizing the following equations to calculate rates of average yield loss per plant (LR1) and (LR2) of each accession of germplasm in the first and second stages of the late sowing:

$$\begin{matrix}LR 1 ( \% ) = [ 1 - ( {P}_{LS 1} / {P}_{NS} ) ] \times 100 \% \\ LR 2 ( \% ) = [ 1 - ( {P}_{LS 2} / {P}_{NS} ) ] \times 100 \%\end{matrix}$$

classifying according to the following heat-tolerant classifying standard of Pisum sativum germplasms:
level 1: 0<LR1≤20% and 0<LR2≤20%;
level 2: (0<LR1≤20% and 20%<LR2≤40%) or (20%<LR1≤40% and 0<LR2≤20%);
level 3: 20%<LR1≤40% and 20%<LR2≤40%;
level 4: (20%<LR1≤40% and 40%<LR2≤60%) or (40%<LR1≤60% and 20%<LR2≤40%);
level 5: 40%<LR1≤60% and 40%<LR2≤60%;
level 6: (40%<LR1≤60% and 60%<LR2≤80%) or (60%<LR1≤80% and 40%<LR2≤60%);
level 7: 60%<LR1≤80% and 60%<LR2≤80%;
level 8: (60%<LR1≤80% and 80%<LR2≤100%) or (80%<LR1≤100% and 60%<LR2≤80%);
level 9: 80%<LR1≤100% and 80%<LR2≤100%.

The present invention further discloses a set of SNP markers related to heat tolerance of Pisum sativum developed based on an SNaPshot technology, including 20 SNP markers shown in Table 3.

The present invention further provides peripheral amplification primer sequences and single base extension primer sequences for the 20 SNP markers, as shown in Table 4.

The present invention further provides use of the aforementioned SNP markers related to heat tolerance of Pisum sativum in analysis of genetic diversity and population genetic structure of heat-tolerant and heat-sensitive Pisum sativum germplasms.

The present invention further provides use of the aforementioned SNP markers related to heat tolerance of Pisum sativum in study of heat-tolerant genetic mechanism and heat-tolerant breeding of Pisum sativum

The present invention further provides a method for conducting genetic diversity and population genetic structure analysis by employing the aforementioned SNP markers related to heat tolerance of Pisum sativum, including
1) SNaPshot PCR reaction
firstly conducting heat tolerance screening through the aforementioned steps, and establishing a genetic population comprising heat-tolerant and heat-sensitive Pisum sativum germplasms; and
then conducting peripheral amplification by using DNAs of the population of Pisum sativum germplasms to be tested as PCR templates with each locus being subjected to single amplification, purifying PCR products and then conducting SNaPshot PCR of them by employing single base extension primers, and detecting reaction products of the SNaPshot PCR by capillary electrophoresis via an ABI 3730XL DNA analyzer; and
2) genetic diversity analysis
conducting data analysis of SNP loci by utilizing Gene mapper 4.1, wherein each sample is genotyped according to peaks corresponding to the SNP loci, and the resultant analysis results are a file of an Excel format and a peak map of a PDF format, and calculating genetic diversity parameters of two groups of SNP markers by utilizing PowerMarker 3.25, wherein the genetic diversity parameters includes a number of genotypes (NG), a major allele frequency (MAF), a number of alleles (NA), gene diversity (GD), expected heterozygosity (He) and polymorphic information content (PIC).
3) population genetic structure analysis
firstly calculating genetic composition of the Pisum sativum germplasms by utilizing Structure 2.3.4, and determining an optimal grouping number of genetic subpopulations of them; secondly verifying the analysis result of Structure by utilizing principal coordinate analysis PCoA; and finally constructing a phylogenetic tree by utilizing UPGMA cluster analysis to display the analysis result intuitively. In the present invention, the screened Pisum sativum germplasms are divided into two genetic subpopulations A and B.

The technical effects of the present invention are as follows: in the present invention, the heat-tolerant classifying standard of Pisum sativum germplasms is established for the first time, and the SNaPshot method is introduced into the identification and evaluation of Pisum sativum germplasms, so that a set of SNP markers (20 markers) related to heat tolerance of Pisum sativum based on an SNaPshot technology is developed, which can analyze the genetic diversity and population genetic structure of heat-tolerant and heat-sensitive Pisum sativum germplasms, thereby providing theoretical and practical basis for future study of heat-tolerant genetic mechanism and heat-tolerant breeding of Pisum sativum

### BRIEF DESCRIPTION OF THE DRAWINGS

FIG. 1 shows the temperature change during the heat tolerance screening of Pisum sativum germplasms;

FIG. 2A shows the changes of daily temperature versus the number of days under high temperature stress during the growth period for different sowing dates during the screening of heat tolerance of Pisum sativum germplasms, including the changes of a daily average maximum temperature, a daily minimum temperature and an average temperature;

FIG. 2B shows the changes of daily temperature versus the number of days under high temperature stress during the growth period for different sowing dates during the screening of heat tolerance of Pisum sativum germplasms, including the days under high temperature stress in the growth period of Pisum sativum; “*” is significant at the level of 0.05; and “**” is significant at the level of 0.01;

FIG. 3A shows the distribution of field emergence rates at normal sowing dates;

FIG. 3B shows field survival rates at different sowing dates of Pisum sativum germplasms;

FIG. 4A shows the distribution of average yield per plant of Pisum sativum germplasms at different sowing dates;

FIG. 4B shows the number of germplasms in respective levels after the heat tolerance screening;

FIG. 5A shows the ΔK in the analysis of the SNaPshot markers related to heat tolerance of Pisum sativum germplasms by Structure;

FIG. 5B shows the ΔK in the analysis of the SNaPshot markers related to heat tolerance of Pisum sativum germplasms by Structure;

FIG. 5C shows the ΔK in the analysis of the SNaPshot markers related to heat tolerance of Pisum sativum germplasms by Structure;

FIG. 5D shows the ΔK in the analysis of the SNaPshot markers related to heat tolerance of Pisum sativum germplasms by Structure;

FIG. 6A shows the population genetic structure of the SNaPshot markers related to heat tolerance in 432 accessions of Pisum sativum germplasms that have been screened for heat tolerance, including analysis of 20 SNaPshot markers related to heat tolerance by Structure;

FIG. 6B shows the population genetic structure of the SNaPshot markers related to heat tolerance in 432 accessions of Pisum sativum germplasms that have been screened for heat tolerance, including PCoA of 20 SNaPshot markers related to heat tolerance;

FIG. 6C shows a genetic distance based on Nei and an UPGMA phylogenetic tree of 20 SNaPshot markers related to heat tolerance;

FIG. 7A shows the population genetic composition of 432 accessions of Pisum sativum germplasms that have been screened for heat tolerance; wherein the population genetic composition is the genetic composition of heat-sensitive Pisum sativum germplasms (n=175) based on 20 SNaPshot markers related to heat tolerance; and

FIG. 7B shows the population genetic composition of 432 accessions of Pisum sativum germplasms that have been screened for heat tolerance; wherein the population genetic composition is the genetic composition of heat-tolerant Pisum sativum germplasms (n=257) based on 20 SNaPshot markers related to heat tolerance.

### DESCRIPTION OF THE EMBODIMENTS

### Example 1

### 1. Materials and Methods

### 1.1. Plant Materials

2,358 accessions of Pisum sativum germplasms from the National Crop Gene Bank of the Institute of Crop Science, Chinese Academy of Agricultural Sciences (Beijing, China) were selected as test materials, of which 1973 accessions (83.67%) were from 29 provinces, cities and autonomous regions in China, 337 accessions (14.29%) were from 25 countries and organizations other than China, and the remaining 48 accessions (2.04%) were of unknown origin and were classified into the category of “unknown”. All Pisum sativum germplasms were divided into two categories according to the type of sowing date, wherein 1,324 accessions (56.15%) were of spring sowing type, and 1,034 accessions (43.85%) were of winter sowing type (Table 1).

TABLE 1
Sources and types of sowing date of 2,358 accessions of Pisum sativum germplasms

| | | Type of sowing date | |
|---|---|---|---|
| Source | Number (accession) | Spring sowing | Winter sowing |
| Shaanxi, China | 257 | 5 | 252 |
| Inner Mongolia, China | 237 | 236 | 1 |
| Qinghai, China | 178 | 178 | |
| Hubei, China | 174 | 8 | 166 |
| Sichuan, China | 173 | 4 | 169 |
| Shanxi, China | 144 | 143 | 1 |
| Gansu, China | 117 | 113 | 4 |
| Xinjiang, China | 103 | 98 | 5 |
| Henan, China | 90 | 48 | 42 |
| Guizhou, China | 78 | 6 | 72 |
| Anhui, China | 75 | 7 | 68 |
| Chongqing, China | 65 | | 65 |
| Yunnan, China | 49 | 21 | 28 |
| Tibet, China | 41 | 41 | |
| Guangxi, China | 37 | 1 | 36 |
| Jiangxi, China | 28 | 1 | 27 |
| Ningxia, China | 25 | 25 | |
| Jiangsu, China | 20 | 3 | 17 |
| Hunan, China | 18 | 2 | 16 |
| Liaoning, China | 17 | 16 | 1 |
| Shanghai, China | 12 | 12 | |
| Hebei, China | 11 | 11 | |
| Beijing, China | 8 | 5 | 3 |
| Guangdong, China | 8 | 4 | 4 |
| Taiwan, China | 3 | 3 | |
| Zhejiang, China | 2 | 1 | 1 |
| Fujian, China | 1 | 1 | |
| Heilongjiang, China | 1 | 1 | |
| Shandong, China | 1 | 1 | |
| Total number in China | 1973 | 995 | 978 |
| United States | 128 | 106 | 22 |
| Germany | 46 | 45 | 1 |
| United Kingdom | 28 | 17 | 11 |
| Nepal | 19 | 18 | 1 |
| Bulgaria | 13 | 13 | |
| France | 11 | 10 | 1 |
| IGARDA | 10 | 10 | |
| Japan | 10 | 8 | 2 |
| Syria | 9 | 8 | 1 |
| Canada | 7 | 7 | |
| Russian Federation | 8 | 7 | 1 |
| Hungary | 7 | 7 | |
| New Zealand | 6 | 6 | |
| Australia | 5 | 4 | 1 |
| Poland | 5 | 5 | |
| Czech | 5 | 5 | |
| Turkey | 5 | 4 | 1 |
| India | 4 | 4 | |
| Denmark | 3 | 3 | |
| Chile | 3 | 3 | |
| Egypt | 1 | 1 | |
| Ethiopia | 1 | 1 | |
| Netherlands | 1 | | 1 |
| Sudan | 1 | | 1 |
| Greece | 1 | | 1 |
| Total number in foreign countries | 337 | 292 | 45 |
| Unknown | 48 | 37 | 11 |
| Total number of spring sowing | | 1324 | |
| Total number of winter sowing | | | 1034 |
| Total number | 2358 | | |

### 1.2 Design of Heat Tolerance Screening Test

This study was carried out in 2017 at a testing farm in Nonggao District, Dongying City, Shandong Province, China (37.258614° north latitude, 118.632774° east longitude, and at an elevation of 14 m). The 2,358 accessions of germplasms were sown in three sowing dates: (1) normal sowing (NS) on March 1; (2) sowing in a first stage of late sowing (LS1) on March 16; and (3) sowing in a second stage of late sowing (LS2) on March 31, wherein the purpose of the latter two stages of late sowing was to apply heat stress during a reproductive phase. 10 seeds were sown per accession of Pisum sativum germplasm at each sowing date, with a row spacing of 0.5 m and a plant spacing of 0.1 m. Irrigation was carried out before sowing to conduct soil moisture generation to meet the requirements of emergence of seedlings. 1.8% EC (5,000×) abamectin was sprayed at emergence and flowering periods to control phytomyza horticola goureau, and weeds were removed by hand. The 2,358 accessions of Pisum sativum germplasms of each sowing date were artificially sown in a completely random arrangement. The field emergence number of each accession of Pisum sativum germplasm in NS, and the number of surviving plants at a maturation period of each accession of Pisum sativum germplasm in NS, LS1 and LS2 were recorded.

### 2.3 Meteorological Data During Heat Tolerance Screening

The test site, Dongying, was located in the northeastern part of the North China Plain, with a temperate continental monsoon climate with four distinct seasons, hot and rainy in summer and cold and dry in winter. All meteorological data during the test were downloaded from the website of Shandong Meteorological Bureau (http://sd.cma.gov.cn/). 2017 was a normal year, and the number of days with a daily average temperature above 22° C. from March to June 2017 in Dongying, Shandong Province, China was 45, which was an ideal condition for distinguishing heat-tolerant and heat-sensitive germplasms.

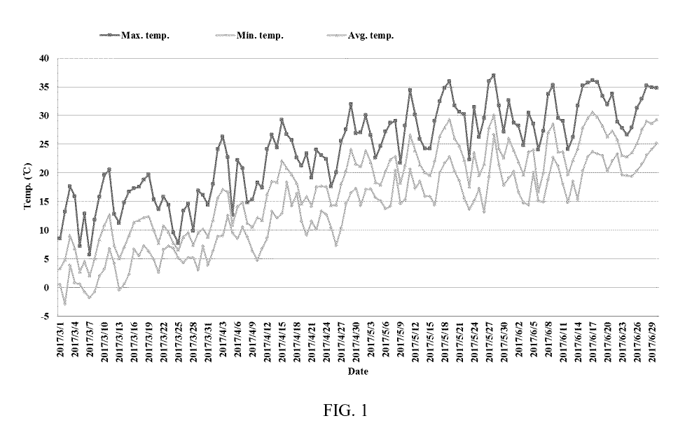

Temperature change data from Mar. 1 to Jun. 30, 2017 were recorded during the heat tolerance screening (FIG. 1). According to the definition of thermal unit by Awasthi et al.: a thermal unit refers to the sum of the average temperatures of all preceding days before the start or completion of a particular phase. This indicator could reflect the degree of the suffered high temperature stress intuitively. The daily maximum temperature, minimum temperature and average temperature variation range and thermal unit accumulation in vegetative and reproductive periods of each sowing date for heat tolerance screening were shown in Table 2. It could be found that the temperature and thermal unit accumulation after delayed sowing were significantly higher than those of normal sowing.

TABLE 2
Comparison of meteorological indicators for heat
tolerance screening at different sowing dates

| Sowing | | Maximum | Minimum | Average | Thermal unit |
|---|---|---|---|---|---|
| date | Growth period | temperature (° C.) | temperature (° C.) | temperature (° C.) | accumulation |
| NS | Vegetative | 5.7-29.2 | −2.9-18.4 | 2-22.1 | 1683.4 |
| | period | | | | |
| | Reproductive | 17.6-37 | 7.3-26.6 | 14.2-27 | |
| | period | | | | |
| LS1 | Vegetative | 7.7-32 | 2.6-18.4 | 6.5-22.1 | 1847 |
| | period | | | | |
| | Reproductive | 21.7-37 | 13.1-26.6 | 16.7-30.6 | |
| | period | | | | |
| LS2 | Vegetative | 12.6-36 | 3.9-22.8 | 8.8-22.2 | 1963.6 |
| | period | | | | |
| | Reproductive | 22.3-37 | 13.1-26.6 | 16.7-30.6 | |
| | period | | | | |

### 2.4 Classification Standard for Heat Tolerance Screening

5 mature plants in each accession of germplasm at the normal sowing date (statistics was not made if the number of adult plants was less than 5), the yield per plant of each plant was weighed, and the average yield per plant (P_{NS}) of the plants was calculated. Corresponding germplasm materials (0-5 plants) in the first and second stages of late sowing were selected, and the average yield per plant (P_{LS1} and P_{LS2}) of the first and second stages of late sowing was calculated. The following equations were utilized to calculate rates of average yield loss per plant (LR1) and (LR2) of each accession of germplasm in the first and second stages of the late sowing:

$$\begin{matrix}LR 1 ( \% ) = [ 1 - ( {P}_{LS 1} / {P}_{NS} ) ] \times 100 \% \\ LR 2 ( \% ) = [ 1 - ( {P}_{LS 2} / {P}_{NS} ) ] \times 100 \%\end{matrix}$$

The heat-tolerant classifying standard of Pisum sativum germplasms was as follows:
level 1:0<LR1≤20% and 0<LR2≤20%
level 2: (0<LR1≤20% and 20%<LR2≤40%) or (20%<LR1≤40% and 0<LR2≤20%)
level 3: 20%<LR1≤40% and 20%<LR2≤40%
level 4: (20%<LR1≤40% and 40%<LR2≤60%) or (40%<LR1≤60% and 20%<LR2≤40%)
level 5: 40%<LR1≤60% and 40%<LR2≤60%
level 6: (40%<LR1≤60% and 60%<LR2≤80%) or (60%<LR1≤80% and 40%<LR2≤60%)
level 7: 60%<LR1≤80% and 60%<LR2≤80%
level 8: (60%<LR1≤80% and 80%<LR2≤100%) or (80%<LR1≤100% and 60%<LR2≤80%)
level 9: 80%<LR1≤100% and 80%<LR2≤100%

Note: if the difference between LR1 and LR2 was higher than 40 yuan %, it will not be included in the heat-tolerant classification.

As mentioned above, according to the magnitude of the numerical value of the average yield loss rate per plant, the heat tolerance of partial Pisum sativum germplasms could be divided into 9 levels, wherein levels 1 to 3 were classified as heat-tolerant (HT) and levels 7 to 9 were classified as heat-sensitive (HS).

### 2.5 SNaPshot Analysis

Genome DNAs were derived from 432 accessions of Pisum sativum germplasms (432 accessions in total of heat-tolerant and heat-sensitive germplasms) after heat tolerance screening. The tender leaves of 3 plants were collected from each accession of material at 4 weeks, and mixed and extracted by a TSINGKE plant DNA extraction kit (Tsingke Biotechnology Co., Ltd., Beijing).

The design of peripheral primers followed the following principle: the primer length was 15-30 bp, and its effective length was generally no more than 38 bp. The GC content should be in 40%-60%, and the optimum Tm value should be in 58-60° C. The primer itself could not contain a self-complementary sequence. There should be no more than 4 complementary or homologous bases between the primers, and especially the complementary overlap at the 3′ terminal should be avoided.

The design principle of single base extension primer: the primer had a length of 15-30 bp, a GC content of 40%-60%, and an optimum Tm value of 58-60° C. PolyCs or PolyTs of different lengths were added to 5′ terminal of the primers, so that each primer could be distinguished by length. The shortest design of the tailed primer was 36 bp, and the lengths of the primers of two adjacent SNP loci generally differed by 4-6 nucleotides.

20 SNP loci related to heat shock proteins or heat shock transcription factors were selected from them by utilizing a GenoPea 13.2K SNP chip developed by Tayeh et al. For each SNP locus sequence, a pair of peripheral amplification primers and one single base extension primer were designed by utilizing Premier 5. See Tables 3 and 4 for SNP loci and SNaPshot primer information.

TABLE 3
SNP locus information

| | | | | Posi- | Predicted | | Predicted |
|---|---|---|---|---|---|---|---|
| Serial | Marker | | | tion | SNP | Predicted | protein |
| Number | Name | Sequence | LG | (cM) | position | SNP effect | function |
| 1 | PsCam054 | ACCAACCACTGATATCCTAACAGCACTT | 1 | 14.7 | Protein | Synonymous | Heat shock |
| | 965_3621 | TCCGAGGACAATCAAAGACTAAGAAGA | | | coding | substitution | transcription |
| | 8_1447 | AAGAA[T/C]CTAATGCTATTATCAGAACT | | | sequence | | factor (HSFA) |
| | | CACTCACATGAAGAATCTCTACAATGAC | | | | | |
| | | ATCATATATTTC | | | | | |
| 2 | PsCam040 | TTATGCGATTTAGATGCTGAATCTTTAGG | 1 | 30.9 | Protein | Nonsynonymous | Heat shock |
| | 389_2514 | AACAATAACAGTGAGAACACCATTTTC | | | coding | substitution | protein |
| | 3_344 | AACA[T/C]GTGCTTTAATCTGATCCAATT | | | sequence | | (hsp17.9) |
| | | TCACATTCTCAGGCAATTCAATCATCCTT | | | | | |
| | | GACAAACCCT | | | | | |
| 3 | PsCam046 | CCTCCAGAAACCGAAAAAACCAACGCC | 1 | 80.3 | of gene; | | Heat shock |
| | 167_2968 | AACGGCAACAACTCTCCTTCAAACTCC | | | non-protein | | transcription |
| | 2_1689 | GACGAC[A/G]TCAACGGAGTCGGATCCA | | | coding | | factor (HSFA) |
| | | CCTCCACAGTCCGATCAACCTCTTCCTC | | | sequence | | |
| | | CAATTCAAAAAACC | | | | | |
| 4 | PsCam045 | ATTTGAGGTAGTGAATGATCATAGTACC | 2 | 34.3 | Protein | Synonymous | Heat shock |
| | 549_2920 | AATCATGTAGTTTCATGGAGCAGAGGTG | | | coding | substitution | transcription |
| | 7_370 | GCAC[T/C]AGCTTTGTGATTTGGGATCTA | | | sequence | | factor (HSFA) |
| | | CATGCTTTCTCCAATGATCTCCTTCCCAG | | | | | |
| | | ATACTTCAAA | | | | | |
| 5 | PsCam020 | TTTGAATCCGATTATATCATGGAGTTGTA | 2 | 44.1 | Protein | Synonymous | Heat shock |
| | 968_1172 | ATGGTGCTAGCTTCGTTGTGTGGGACCC | | | coding | substitution | transcription |
| | 2_484 | TTT[A/G]GAGTTTGCTAGAATCATTTTGC | | | sequence | | factor (HSFA) |
| | | CTCGACATTTCAAACACAACAATTTCTC | | | | | |
| | | CAGTTTTGTT | | | | | |
| 6 | PsCam050 | TGGAAGCTAGGTTGTTGATTACTGAGAA | 3 | 24.5 | Protein | Nonsynonymous | Heat shock |
| | 106_3271 | GAAACATCAACAGATGATGGCTTTTCTT | | | coding | substitution | transcription |
| | 1_1241 | GCAA[A/G]AGCACTCAGTAATCAATCTT | | | sequence | | factor (HSFA) |
| | | TTATTCAGCAATTGGCAAACAACAAAG | | | | | |
| | | AGTTGAAAGGTGT | | | | | |
| 7 | PsCam039 | CGTAGGAAATCGATTATCCCATATGGGT | 3 | 51.7 | Protein | Synonymous | HS21C_Pisum |
| | 017_2400 | GTGGAAGGAACAAGTCTCTTTTTGGGT | | | coding | substitution | sativum |
| | 3_766 | TGAGG[T/C]TGGTGCGGTTTGTTGCTTG | | | sequence | | RecName: |
| | | AACTTGAGACATGGTCCAGTTTTTCTCT | | | | | Full = small |
| | | TGCGTCGCCTGTC | | | | | heat shock |
| | | | | | | | protein, |
| | | | | | | | chloroplastic; |
| | | | | | | | Flags: |
| | | | | | | | Precursor |
| 8 | PsCam049 | GAATAGGATACCAATTACCTTCTGTTTCT | 3 | 131.1 | Protein | Synonymous | Heat shock |
| | 061_3170 | CATTGATGTCAATGTGATCATATTTGGGA | | | coding | substitution | protein hsp70 |
| | 5_537 | TC[A/G]TTAGACATTGCAACTTCTCTGTA | | | sequence | | |
| | | ACTATTGATACAATAGACAAGCTGCTCA | | | | | |
| | | ATTACTGCA | | | | | |
| 9 | PsCam038 | AACAGAATTTTCACTCAATAATTTCTCT | 4 | 14.4 | Protein | Synonymous | Heat shock |
| | 211_2325 | GACATAACATGATAAGCAGAAGAAACA | | | coding | substitution | transcription |
| | 6_428 | TCTTG[A/C]GCACTTTGTCTAGAATCACC | | | sequence | | factor (HSFA) |
| | | GCAAGCCCTCATTTCATGTAAAGAGCTA | | | | | |
| | | ACGAATCCATTC | | | | | |
| 10 | PsCam027 | ATCCACATGCGCAGACCATATTTTAGCC | 4 | 36.5 | of gene; | | Heat shock |
| | 754_1632 | ATTATGTGCCTAATACTCATAGCCAACGG | | | non-protein | | transcription |
| | 1_1079 | GCG[A/G]CAACTTCTGGTGTTAGTGTTG | | | coding | | factor (HSFA) |
| | | CTGCTGCTCCATTAACAGAATCAGAGCG | | | sequence | | |
| | | GTGGAATTTTA | | | | | |
| 11 | PsCam001 | AACGTTGATGTTTATCGTCACTCCTTCC | 4 | 75.1 | of gene; | | Heat shock |
| | 594_1320 | CTCGCACTCGCCGCGATGATCTTCTTCT | | | non-protein | | protein |
| | 450 | CTCA[A/G]GTAGTTCTTATCGCTCTCTTT | | | coding | | |
| | | TGTTATGTTGATTTGATTCACTAGTATAT | | | sequence | | |
| | | AATATAAATT | | | | | |
| 12 | PsCam059 | GGGGAAATATGTGTGGAAAGCGTTTAA | 5 | 43.6 | of gene; | | Heat shock |
| | 391_3958 | ATTTTGGTGTTTCTAACTCTTATAATATAT | | | non-protein | | transcription |
| | 2_1896 | GTG[T/C]CGTTTTGTTGTAGTAACAGGTT | | | coding | | factor (HSFA) |
| | | GAAACATTGGTTTCTCATGTTTGTGATC | | | sequence | | |
| | | TCTTTGATAT | | | | | |
| 13 | PsCam039 | TTAATTTGAATTAGATATGGGAAGGGAC | 5 | 49.6 | of gene; | | Heat shock |
| | 062_2404 | CTTAAGATATAAAATAATTTGTCCCTATT | | | non-protein | | transcription |
| | 4_1315 | CAA[T/G]ACTATTACAATTCAACCACATA | | | coding | | factor (HSFA) |
| | | TAAAATTCAATGTTAGTAGGCGACAGAC | | | sequence | | |
| | | ATGTCTTAGA | | | | | |
| 14 | PsCam013 | AAACAATCGTGCAGAGATCATTCATAAT | 5 | 92.2 | Protein | Synonymous | Heat shock |
| | 828_9409 | GATCAAGGAAACAGAACTACACCTTCT | | | coding | substitution | protein hsp70 |
| | 206 | TTTGT[T/C]GCTTTTACTGATTCTCAAAG | | | sequence | | |
| | | ATTGATTGGCGATGCTGCAAAAAATCAG | | | | | |
| | | GCTGCTTCAAAC | | | | | |
| 15 | PsCam057 | TTTAGTGATTCTGTTATTCAAAATGATTT | 5 | 92.3 | Protein | Nonsynonymous | Heat shock |
| | 184_3783 | GAATTTGTGGCCATTCAAAGTCATTTCT | | | coding | substitution | protein hsp70 |
| | 1_137 | GGT[A/G]TCCATGACAAACCCATGATTG | | | sequence | | |
| | | TTGTTCAATACAAGGGTGAAGAGAAAC | | | | | |
| | | ACTTTTGTGCTG | | | | | |
| 16 | PsCam036 | AGAGAAAGTGAGAGAAAAATAAAGAA | 6 | 49.6 | of gene; | | Heat shock |
| | 720_2184 | AGGAAGGAACGAGAAGGTACTGCGTTA | | | non-protein | | transcription |
| | 7_1549 | GTTACGT[T/C]TAATTGTCGAGAAAGTTC | | | coding | | factor (HSFA) |
| | | CGTGAGAATGAGGATTTTCAGTTTGATT | | | sequence | | |
| | | AATGATGATAGAAT | | | | | |
| 17 | PsCam028 | AACAATGAAACTCCTATTTGTAGAACTC | 6 | 87.4 | Protein | Synonymous | Heat shock |
| | 698_1699 | CATGAAATAATGGCATTAGTTGTAGGAT | | | coding | substitution | transcription |
| | 9_180 | CATT[T/G]ACCATCATATAAATCTTACAC | | | sequence | | factor (HSFA) |
| | | A | | | | | |
| 18 | PsCam045 | GAAGGATAGAGAGAGCATGGCACGAGC | 7 | 13.9 | of gene; | | Heat shock |
| | 359_2904 | CTCTCTAATTGTTCTCGCCATTATTTCAA | | | non-protein | | protein hsp70 |
| | 3_269 | TAGG[T/C]AAGTTTTTCGCTGCTTATTAT | | | coding | | |
| | | TAGATCTTATGGTTTAATGCCGAAAATGT | | | sequence | | |
| | | AACCGATTGA | | | | | |
| 19 | PsCam035 | TTGCTTTCCCCATATTCCACCAAAATCTT | 7 | 34.9 | Protein | Synonymous | Heat shock |
| | 563_2074 | CCGTGACCACTAGGTTTTCCTTCATTTTT | | | coding | substitution | transcription |
| | 1_363 | TC[T/G]GCTATTATTATCCTTTCTTTCT | | | sequence | | factor (HSFA) |
| | | GA | | | | | |
| | | TTGTACTTCCTGTGCTTCTGCCGCACCA | | | | | |
| | | GGATCCTC | | | | | |
| 20 | PsCam042 | ATAGGTATTCAACTGCCTGACAAAGCTG | 7 | 69.4 | Protein | Synonymous | Heat shock |
| | 861_2690 | GAGAAGTTATTGTGCTTAAAATACTTAG | | | coding | substitution | transcription |
| | 1_2929 | GCAA[A/G]ATATATTTGGAGAAATCGGT | | | sequence | | factor (HSFA) |
| | | GGCATTCAAAACGACAAAGGTGTTATT | | | | | |
| | | GTTTTTTCCCCAC | | | | | |

TABLE 4
SNaPshot primer information

| | | | | Names | |
|---|---|---|---|---|---|
| | | | | of SNP | |
| | | Names of | Sequences of | single | Sequences of SNP |
| | | peripheral | peripheral | base | single base |
| Serial | Marker | amplification | amplification | extension | extension |
| Number | Name | primers | primers | primers | primers |
| 1 | PsCam054965_ | 1-F | GAACAATCACCTCAAAT | 1-SNP-F | TTTTTTCAATCAAAGACTAAGAA |
| | 36218_ | | CCTCCA | | GAAAGAA |
| | 1447 | 1-R | ACCGATGAATCCAACTC | | |
| | | | AACAAG | | |
| 2 | PsCam040389_ | 2-F | TAACCGCCAAGATCAAG | 2-SNP-F | TTTTTTTTTTTTTTTTTTTTTTTAG |
| | 25143_ | | GGA | | TGAGAACACCATTTTCAACA |
| | 344 | 2-R | GCTGAGAGAGGGACTGG | | |
| | | | AAA | | |
| 3 | PsCam046167_ | 3-F | TCCTCACCGAAATCAAA | 3-SNP-F | TTTTTTTTTTTTTTTTTCCTTCAAA |
| | 29682_ | | CGC | | CTCCGACGAC |
| | 1689 | 3-R | AAAGACACCAACTCATC | | |
| | | | GCACT | | |
| 4 | PsCam045549_ | 4-F | AACAAGCATTGATTCTAC | 4-SNP-F | TTTTTTTTTTTTTTTTTTTTTTATG |
| | 29207_ | | CACCC | | GAGCAGAGGTGGCAC |
| | 370 | 4-R | ACAAACAACAAAAGCA | | |
| | | | AGCCG | | |
| 5 | PsCam020968_ | 5-F | GCCTATGCCTATGGAGTG | 5-SNP-F | TTTTTTTTTTTTTTTTTTTTTTTTTT |
| | 11722_ | | TTTG | | TCGTTGTGTGGGACCCTTT |
| | 484 | 5-R | TTACATTTAGAGGGTGAC | | |
| | | | AAAGCAA | | |
| 6 | PsCam050106_ | 6-F | CAACGAGAAAGTGGTGG | 6-SNP-F | TTTTTTTTTTTTTTTTTTTTTTTTTT |
| | 32711_ | | TGC | | TTTTATGATGGCTTTTCTTGCAA |
| | 1241 | 6-R | AGCCTCCTCTTTCGCTTC | | |
| | | | AT | | |
| 7 | PsCam039017_ | 7-F | TCCTTCTTTAATCTCCCA | 7-SNP-R | TTTTTTTTTTTTTTTTTTTTTTTTTT |
| | 24003_ | | CGG | | TTTTTTTTTAAGCAACAAACCGCA |
| | 766 | 7-R | ATTGAAACCAACTCACT | | CCA |
| | | | TCCCTT | | |
| 8 | PsCam049061_ | 8-F | AAGCCAGTTCTCAGCTT | 8-SNP-F | TTTTTTTTTTTTTTTTTTTTTTTTTT |
| | 31705_ | | CTCC | | TTTTTCAATGTGATCATATTTGGG |
| | 537 | 8-R | TCGAGGAGCGCTACAAA | | ATC |
| | | | GAA | | |
| 9 | PsCam038211_ | 9-F | TCCAACCAAACCACTAG | 9-SNP-R | TTTTTTTTTTTTTTTTTTTTTTTTTT |
| | 23256_ | | CAGATC | | TTTTTTTTTTCGGTGATTCTAGAC |
| | 428 | 9-R | AGGCCTCAAGGTCGAGA | | AAAGTGC |
| | | | ACAG | | |
| 10 | PsCam027754_ | 10-F | TGATCAGGGCTTTAGAA | 10-SNP-R | TTTTTTTTTTTTTTTTTTTTTTTTTT |
| | 16321_ | | AGGTTG | | TGCAACACTAACACCAGAAGTTG |
| | 1079 | 10-R | CACTCTTCTTGCTGCCTC | | |
| | | | TGAC | | |
| 11 | PsCam001594_ | 11-F | TCAAGCTCTTTTCTTCGC | 11-SNP-R | TTTTTTTTTTTTTTTTTTTTTTTTTT |
| | 1320_ | | CC | | TTTTTTTTTTTACAAAAGAGAGCG |
| | 450 | 11-R | CTGTCTCTCTCGCATCCC | | ATAAGAACTAC |
| | | | ATC | | |
| 12 | PsCam059391_ | 12-F | TGGAAACTTGGTGTGAC | 12-SNP-R | TTTTTTTCAACCTGTTACTACAAC |
| | 39582_ | | AGTAAATC | | AAAACG |
| | 1896 | 12-R | TCAACTATCAGAGATTCT | | |
| | | | TGTCCAA | | |
| 13 | PsCam039062_ | 13-F | AGCCATTTGTTTTGTTTT | 13-SNP-F | TTTTTTTTTTTATAAAATAATTTGT |
| | 24044_ | | CTTGAG | | CCCTATTCAA |
| | 1315 | 13-R | TCATTGCAAAATTCCCAT | | |
| | | | CTACTT | | |
| 14 | PsCam013828_ | 14-F | TGTGTAGCAGTTTGGCA | 14-SNP-F | TTTTTTAAACAGAACTACACCTTC |
| | 9409_ | | GGG | | TTTTGT |
| | 206 | 14-R | AGGTTTGTCATCTTTGCC | | |
| | | | AGC | | |
| 15 | PsCam057184_ | 15-F | GCTTCTAACCCAACCAAT | 15-SNP-F | TTTTTTTTTTTTTTTTTTTCCATTC |
| | 37831_ | | ACAGTC | | AAAGTCATTTCTGGT |
| | 137 | 15-R | ACTTCACGCATCTTTCTG | | |
| | | | AGGAC | | |
| 16 | PsCam036720_ | 16-F | ATAGGGGGTGTTATTGTG | 16-SNP-F | TTTTTTTTTTTTTTTTTTTTTTAGA |
| | 21847_ | | GTGAT | | AGGTACTGCGTTAGTTACGT |
| | 1549 | 16-R | GATTGCGAAGAGCCGTG | | |
| | | | AA | | |
| 17 | PsCam028698_ | 17-F | AACTCGAGAAATTGTTG | 17-SNP-F | TTTTTTTTTTTTTTTTTTTTTTTTTT |
| | 16999_ | | TGCCTA | | TTGGCATTAGTTGTAGGATCATT |
| | 180 | 17-R | TGAGATTGTGCTACTATG | | |
| | | | GAGCAA | | |
| 18 | PsCam045359_ | 18-F | TCACCGTCTTCACCATTA | 18-SNP-F | TTTTTTTTTTTTTTTTTTTTTTTTTT |
| | 29043_ | | CACC | | TTTTTTTCTCGCCATTATTTCAATA |
| | 269 | 18-R | ACAGTTGAACAAAACCT | | GG |
| | | | GATCCA | | |
| 19 | PsCam035563_ | 19-F | GGCATCACAACCAGAAG | 19-SNP-F | TTTTTTTTTTTTTTTTTTTTTTTTTT |
| | 20741_ | | AAATAGT | | TTTTTTATCAGAAAGAAAGGATAA |
| | 363 | 19-R | GCCTGTAGGAATCACCA | | TAATAGC |
| | | | CTGC | | |
| 20 | PsCam042861_ | 20-F | GGTATTCAACTGCCTGAC | 20-SNP-F | TTTTTTTTTTTTTTTTTTTTTTTTTT |
| | 26901_ | | AAAGC | | TTTTTTTTTTTTTTGTGCTTAAAAT |
| | 2929 | 20-R | CTACGACATGGTGGATG | | ACTTAGGCAA |
| | | | ACTCTTC | | |

The extracted DNA sample was diluted to 20 ng/μl and then used as a PCR template to conduct peripheral amplification with 1.1×T3 Super PCR Mix (Tsingke Biotechnology Co., Ltd., Beijing), wherein each locus was subjected to single amplification, and each pair of primers was amplified according to the following amplification system and procedures. The amplification system was 35 μl in total: 30 μl of 1.1×T3 Super PCR Mix; 2 μl of 10 μM Primer F; 2 μl of 10 μM Primer R; and 1 μl of a Template (gDNA). Amplification procedure: 98° C. for 3 min; 98° C. for 10 s, 57° C. for 10 s, 72° C. for 15 s, 35 cycles; 72° C. for 2 min; storage at 4° C. The amplified PCR product was subjected to agarose gel electrophoresis (2 μl of the sample+6 μl of bromophenol blue) at a voltage of 300 V for 12 minutes, to acquire an identification gel photograph through which the size of a band of interest was determined. The PCR products were purified by utilizing an MagS Magnetic Bead Gel Recovery Kit (Tsingke Biotechnology Co., Ltd., Beijing).

The purified single PCR products were ready for use. The single base extension primers were diluted to 10 μM, and SNaPshot PCR was conducted. The PCR system was 5 μl in total: 2 μl of ABI SnapShot multiplex Mix (Applied Biosystems, Foster City, CA, USA); 1 μl of the Primers; 1 μl of a purified Post-PCR Template; and 1 μl of ddH₂O. Amplification procedure: 96° C. for 2 min; 96° C. for 10 s, 50° C. for 5 s, 60° C. for 30 s, 30 cycles; 60° C. for 30 s; storage at 4° C. The SNaPshot PCR reaction products were detected by capillary electrophoresis via an ABI 3730XL DNA analyzer (Applied Biosystems, Foster City, USA).

### 2.6 Data Analysis

The field emergence rate of each accession of Pisum sativum germplasm in NS was calculated by Excel, according to the equation: field emergence rate (%)=field emergence number/10×100%. The field survival rates of each accession of Pisum sativum germplasm at different sowing dates were calculated by Excel, according to the equation: field survival rate (%)=number of surviving plants at the maturation period/10×100%.

Data analysis of SNP loci was conducted by utilizing Gene mapper 4.1, wherein each sample was genotyped according to peaks corresponding to the SNP loci, and the resultant analysis results were a file of an Excel format and a peak map of a PDF format. The genetic diversity parameters of two groups of SNP markers were calculated by utilizing PowerMarker 3.25, wherein the genetic diversity parameters included a number of genotypes (NG), a major allele frequency (MAF), a number of alleles (NA), gene diversity (GD), expected heterozygosity (He) and polymorphic information content (PIC).

Genetic structure analysis of heat-tolerant SNP markers was carried out on three Pisum sativum populations obtained after heat-tolerance screening by utilizing different population genetic structure analysis methods. Firstly, Bayesian cluster analysis was conducted by utilizing Structure 2.3.4. The parameters were set as follows: Length of Burnin Period=10,000, Number of MCMC Reps after Burnin=100,000, Number of Population=1-10, and Number of Iterations=10. According to the algorithm proposed by Evanno et al., the optimal population structure and population size were determined according to a Delta K (ΔK) value (the online analysis website was http://taylor0.biology.ucla.edu/struct_harvest/). Secondly, principal coordinate analysis (PCoA) was conducted by utilizing GenAlEx 6.5 to check whether the population genetic analysis of the Pisum sativum population obtained after heat tolerance screening was reasonable. Finally, a phylogenetic tree of the three Pisum sativum populations obtained after heat tolerance screening was constructed based on UPGMA (unweighted pair-group method) by utilizing PowerMarker 3.25, and displayed with Figtree 1.4.3 (https://github.com/rambaut/figtree/releases/tag/v1.4.3). Moreover, the significance test of the difference of means and the Kolmogorov-Smirnov test were completed by SPSS 20.0.

### 3. Results

### 3.1 High Temperature Stress During Heat Tolerance Screening

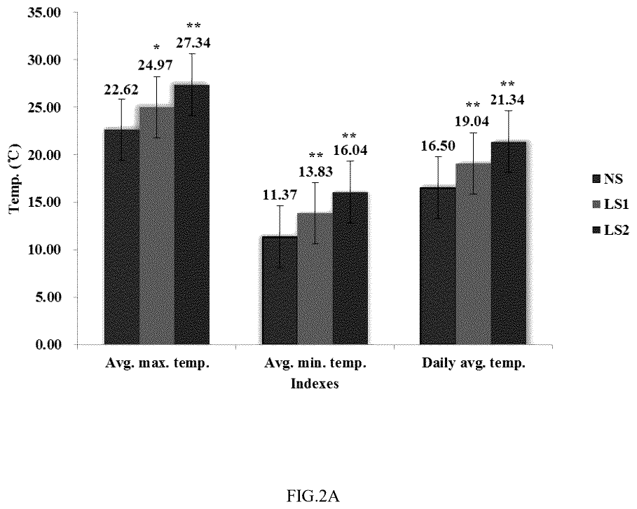

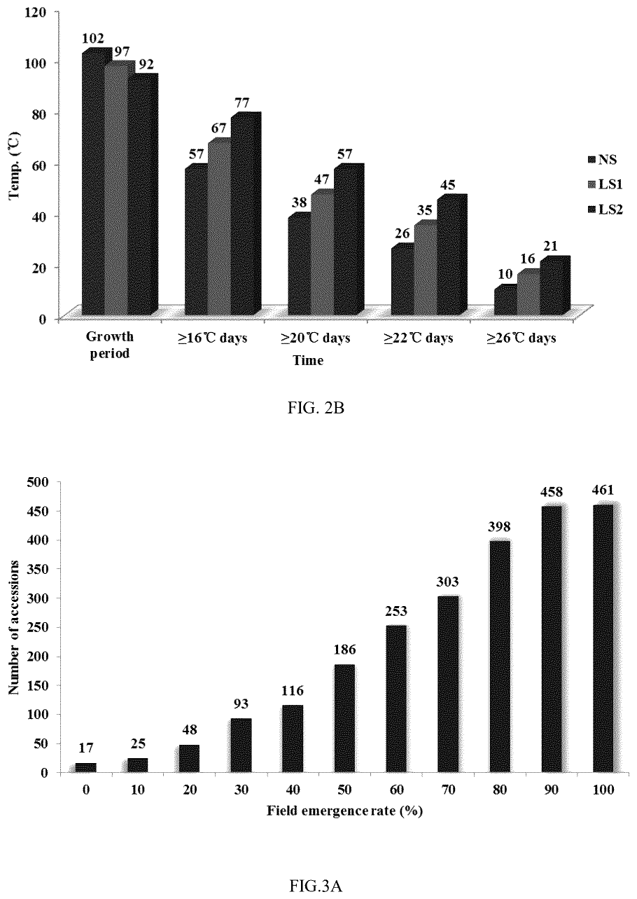

During the heat tolerance screening, the daily average maximum temperature, daily average minimum temperature and average temperature in the growth period of Pisum sativum at different sowing dates all showed an increasing trend (FIG. 2A), and the aforementioned indicators of the first stage of late sowing (LS1) and the second stage of late sowing (LS2) were significantly or extremely significantly higher than those of the normal sowing date (NS). For Pisum sativum, the preferred daily average temperature was 12-16° C. in the vegetative period, 16-20° C. in the flowering period, and 16-22° C. in the pod bearing period, and the metabolic activity tended to stop when the temperature was higher than 26° C. The number of days in which the daily average temperature was higher than 16° C., 20° C., 22° C. and 26° C. in the growth period of Pisum sativum during the heat tolerance screening was shown in FIG. 2B, and the number of days of the growth period at the three sowing dates (NS, LS1 and LS2) was presented with a progressive decreasing trend, while the number of days in which the temperature was higher than the aforementioned nodal temperature was presented with a progressive increasing trend. The aforementioned results showed that the high temperature stress effects of LS1 and LS2 were significant, which met the requirements for heat tolerance screening of Pisum sativum germplasms.

3.2 Field Emergence Rates of Pisum sativum Germplasms at Normal Sowing Dates

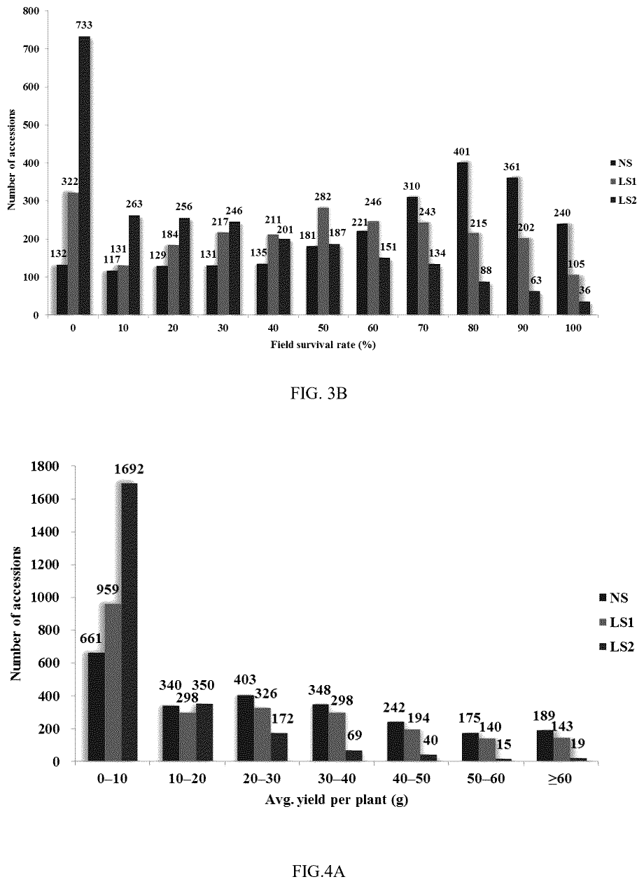

The distribution of field emergence rates of 2,358 accessions of Pisum sativum germplasms at normal sowing dates were shown in FIG. 3A, with an average value of 73.6% and a variation range of 0-100%. Among them, the field emergence rates of 1873 accessions (79.4%) were no less than 60%, and the field emergence rates of 461 accessions (19.6%) were 100%; the field emergence rates of only 485 accessions (20.6%) were lower than 60%, and the field emergence rates of 17 accessions (0.7%) were zero. The results showed that the seed vigor of the Pisum sativum germplasms in the National Crop Germplasm Bank of China was relatively high, which could meet the requirements of this study.

3.3 Field Survival Rates of Pisum sativum Germplasms at Different Sowing Dates

The distribution of field survival rates of Pisum sativum germplasms at different sowing dates was shown in FIG. 2B. The mean of the field survival rates in NS was 61.8%, wherein the field survival rates of 1,533 accessions (65.0%) were no less than 60%, and the number of germplasms with a survival rate of 80% was the largest, reaching 401 accessions (17.0%). The mean of the field survival rates in LS1 was 47.4%, which was lower than that of NS, wherein the field survival rates of 1,011 accessions (42.9%) were no less than 60%, which was significantly lower than that of NS, and the number of germplasms with a survival rate of 0 reached 322 accessions (13.7%). The mean of the field survival rates in LS2 was 28.5%, which was much lower than that of NS, wherein the field survival rates of only 472 accessions (20.0%) were no less than 60%, which was very much lower than that of NS, and the number of germplasms with a survival rate of 0 was up to 733 accessions (31.1%). The results showed that the field survival rates of LS1 and LS2 were lower than that of NS after treatment with high temperature stress, and this was especially obvious in LS2.

3.4 Yield Per Plant of Pisum sativum Germplasms at Different Sowing Dates

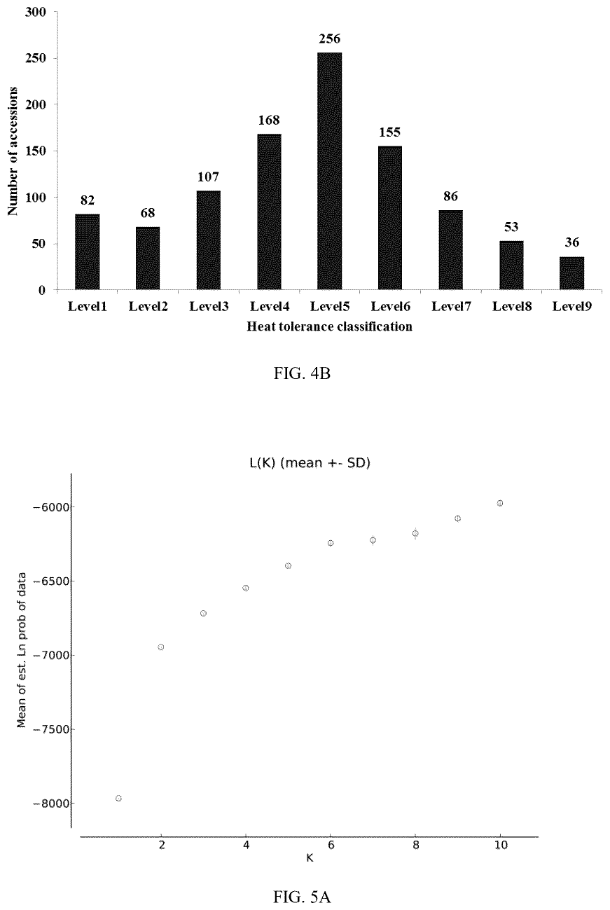

The distribution of average yield per plant of each accession of Pisum sativum germplasm at different sowing dates after heat tolerance screening was shown in FIG. 4A. It could be seen that in NS, the number of germplasms with an average yield per plant between 0-10 g was 661 accessions, accounting for 28.0% of the total number of the experimental materials; the number of germplasms with an average yield per plant higher than 60 g was 189 accessions (8.0%); and the numbers of germplasms in the remaining respective levels were distributed relatively evenly. In LS1, the number of germplasms with an average yield per plant between 0-10 g was 959 accessions (40.7%), which was increased compared with that of NS; and the numbers of germplasms in the remaining respective levels were all decreased compared with that of NS, indicating that high temperature stress had a certain impact on the yield of Pisum sativum germplasms. In LS2, the number of germplasms with an average yield per plant between 0-10 g was up to 1,692 accessions (71.8%), which far exceeded the numbers of germplasms in the corresponding level of NS and in the remaining respective levels, indicating that high temperature stress had an extremely serious impact on the yield of Pisum sativum germplasms.

### 3.5 Number of Germplasms in Respective Levels After Heat Tolerance Screening

The numbers of germplasms in respective levels after the heat tolerance screening was determined according to the classification standard for heat tolerance screening of Pisum sativum germplasms, as shown in FIG. 4B. After the Kolmogorov-Smirnov test of the numbers of germplasms in respective levels, the asymptotic significance probability value P was 0.850>0.05, so it obeyed the normal distribution, indicating that the experimental design of the heat tolerance screening was reasonable and feasible. The numbers of germplasms divided into levels 1, 2 and 3 were 82, 68 and 107 accessions, respectively, and the three ones were collectively referred to as heat-tolerant (HT) germplasms, with a total of 257 accessions; and the numbers of germplasms divided into levels 7, 8 and 9 were 86, 53 and 36 accessions, respectively, and the three ones were collectively referred to as heat-sensitive (HS) germplasms, with a total of 175 accessions, so that there were 432 accessions of heat-tolerant and heat-sensitive germplasms in total.

3.6 Heat Tolerance and Type of Sowing Date of Pisum sativum Germplasms

The 2,358 accessions of Pisum sativum germplasms could be divided into two categories of spring sowing and winter sowing according to the type of sowing date, wherein 1,324 accessions (56.1%) are of the spring sowing type, and 1,034 accessions (43.9%) are of the winter sowing type (Table 1). After heat tolerance screening, there were a total of 246 accessions of the spring sowing type and a total of 186 accessions of the winter sowing type. Among the 257 heat-tolerant germplasms, 100 accessions (38.9%) were of the spring sowing type, and 157 accessions (61.1%) were of the winter sowing type; and among the 175 heat-sensitive germplasms, 146 accessions (83.4%) were of the spring sowing type, and 29 accessions (16.6%) were of the winter sowing type. Among the heat-tolerant germplasms, there were fewer germplasms of the spring-sowing type than those of the winter sowing type, while among the heat-sensitive germplasms, there were much more germplasms of the spring-sowing type than those of the winter sowing type

### 3.7 Genetic Diversity Analysis of the Population Obtained After Heat Tolerance Screening

Genetic diversity evaluation of populations after the heat tolerance screening was conducted by utilizing the SNaPshot markers related to heat tolerance. The total numbers of NG and NA were 52 and 39, respectively (Table 5). The means of MAF, GD, He, and PIC were 0.749, 0.313, 0.156, and 0.246, respectively (Table 5), and the ranges of them were 0.530-1, 0-0.498, 0-0.488, and 0-0.374, respectively (Table 6). According to the magnitude of the PIC value, there were 13 SNaPshot markers with medium PIC and 7 SNaPshot markers with low PIC (Table 5). The analysis results of the SNaPshot markers related to heat tolerance showed that the Pisum sativum germplasm population after the screening had relatively high genetic diversity.

TABLE 5
Summary of genetic diversity parameters of SNP markers in
Pisum sativum germplasm population after heat tolerance screening

| Number of | Total | Total | Average | Average | Average | Average | Type of information (PIC) | | |
|---|---|---|---|---|---|---|---|---|---|
| markers | NG | NA | MAF | GD | He | PIC | Low | Medium | High |
| 20 | 52 | 39 | 0.749 | 0.313 | 0.156 | 0.246 | 7 | 13 | 0 |
| | | | | | | | (35.0%) | (65.0%) | |

note:
NG: the number of genotypes;
NA: the number of alleles;
MAF: major allele frequency;
GD: gene diversity;
He: expected heterozygosity;
PIC: polymorphic information content, high (PIC ≥ 0.5), medium (0.25 ≤ PIC < 0.5), and low (PIC < 0.25).

TABLE 6
Genetic diversity indicators of SNAPshot markers
related to heat tolerance of Pisum sativum

| ID | NG | NA | MAF | GD | He | PIC |
|---|---|---|---|---|---|---|
| 1 | 3 | 2 | 0.544 | 0.496 | 0.370 | 0.373 |
| 2 | 1 | 1 | 1.000 | 0.000 | 0.000 | 0.000 |
| 3 | 3 | 2 | 0.961 | 0.076 | 0.046 | 0.073 |
| 4 | 3 | 2 | 0.795 | 0.326 | 0.294 | 0.273 |
| 5 | 3 | 2 | 0.641 | 0.460 | 0.171 | 0.354 |
| 6 | 3 | 2 | 0.542 | 0.497 | 0.056 | 0.373 |
| 7 | 3 | 2 | 0.757 | 0.368 | 0.162 | 0.300 |
| 8 | 3 | 2 | 0.965 | 0.067 | 0.046 | 0.065 |
| 9 | 3 | 2 | 0.631 | 0.466 | 0.178 | 0.357 |
| 10 | 3 | 2 | 0.588 | 0.485 | 0.218 | 0.367 |
| 11 | 3 | 2 | 0.954 | 0.088 | 0.074 | 0.084 |
| 12 | 3 | 2 | 0.573 | 0.489 | 0.169 | 0.370 |
| 13 | 3 | 2 | 0.878 | 0.214 | 0.118 | 0.191 |
| 14 | 2 | 2 | 0.999 | 0.002 | 0.002 | 0.002 |
| 15 | 3 | 2 | 0.994 | 0.012 | 0.002 | 0.011 |
| 16 | 3 | 2 | 0.558 | 0.493 | 0.204 | 0.372 |
| 17 | 2 | 2 | 0.756 | 0.369 | 0.488 | 0.301 |
| 18 | 3 | 2 | 0.530 | 0.498 | 0.222 | 0.374 |
| 19 | 3 | 2 | 0.758 | 0.367 | 0.160 | 0.300 |
| 20 | 3 | 2 | 0.560 | 0.493 | 0.144 | 0.371 |
| Mean | 2.800 | 1.950 | 0.749 | 0.313 | 0.156 | 0.246 |
| Max | 3 | 2 | 1.000 | 0.498 | 0.488 | 0.374 |
| Min | 1 | 1 | 0.530 | 0.000 | 0.000 | 0.000 |

### 3.8 Genetic Structure Analysis of the Population Obtained After Heat Tolerance Screening

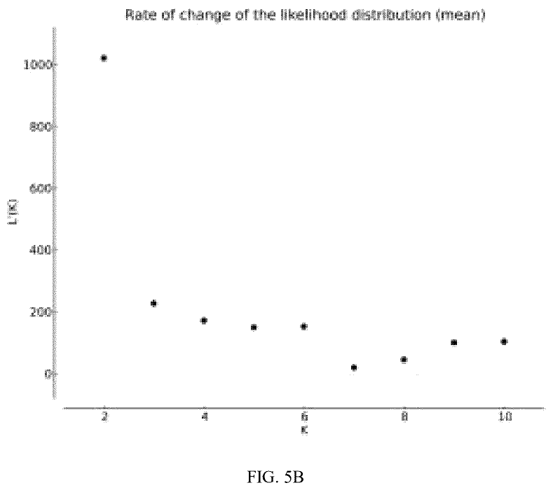

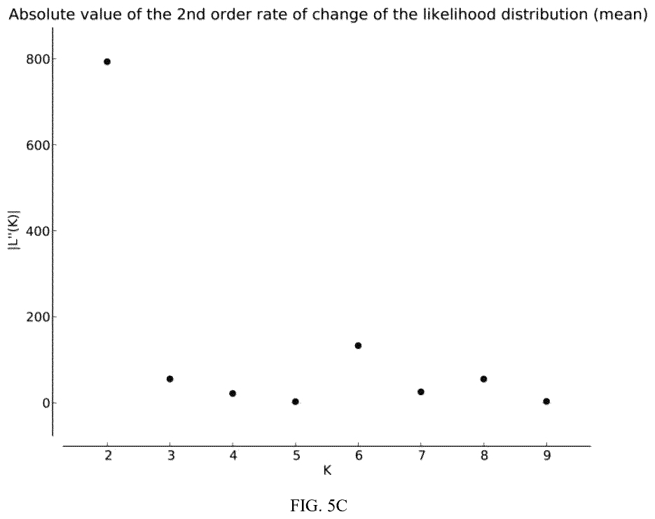

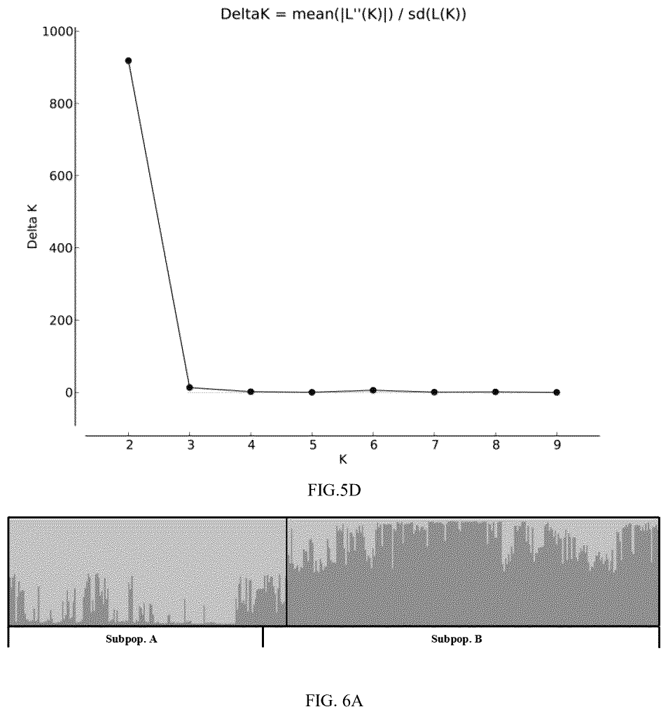

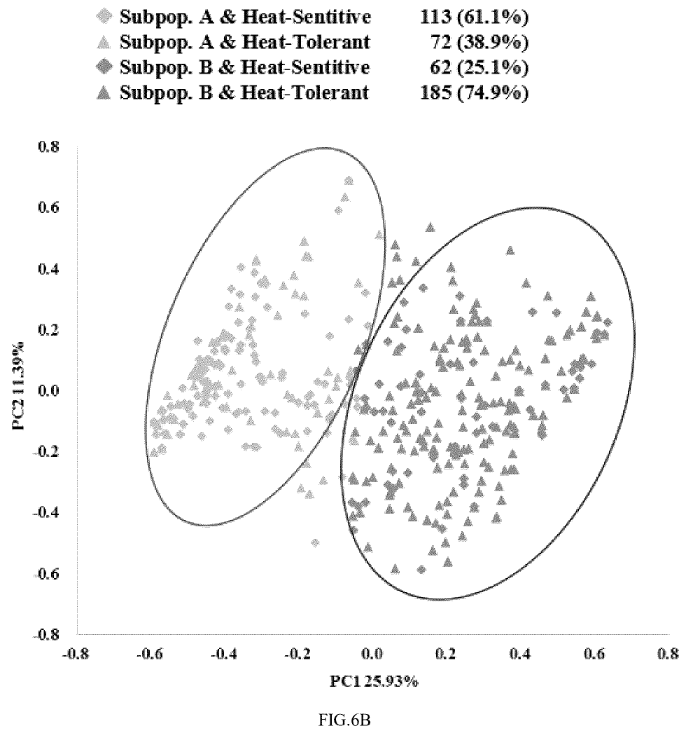

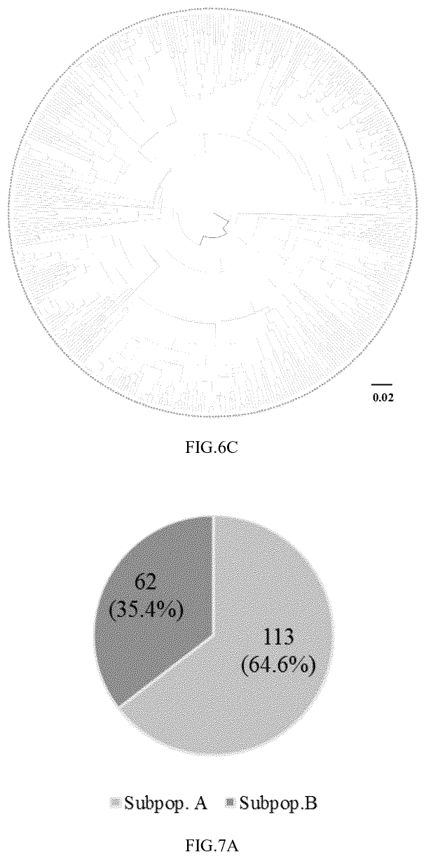

In order to study the population genetic structure of the Pisum sativum germplasm after screening, the genetic composition of 432 accessions of Pisum sativum germplasms was calculated by utilizing Structure 2.3.4, and the optimal grouping number (K) of the genetic subpopulations was determined. The Evanno ΔK value was also the highest when the K of the genetic subpopulations=2 (FIGS. 5A-5D). In FIG. 6A, orange (shown in light gray in a black-and-white diagram) represented subpopulation A, with a total of 185 accessions, including 72 accessions (38.9%) of heat-tolerant germplasms and 113 accessions (61.1%) of heat-sensitive germplasms; in the subpopulation A, there were 126 accessions (68.1%) of the spring sowing type and 59 accessions (31.9%) of the winter sowing type. Light blue (shown in dark gray in a black-and-white diagram) represented subpopulation B, with a total of 247 accessions, including 185 accessions (74.9%) of heat-tolerant germplasms and 62 accessions (25.1%) of heat-sensitive germplasms; in the subpopulation B, there were 120 accessions (48.6%) of the spring sowing type and 127 accessions (51.4%) of the winter sowing type (Table 7).

TABLE 7
Grouping of genetic subpopulations of Pisum sativum germplasms based on Structure
analysis of SNAPshot markers related to heat tolerance

| Heat-tolerant | Subpop. A | | Subpop. B | | | Subpop. A | | Subpop. B | |
|---|---|---|---|---|---|---|---|---|---|
| type of | | Proportion | | Proportion | | | Proportion | | Proportion |
| germplasm | Number | (%) | Number | (%) | Sowing Type | Number | (%) | Number | (%) |
| Heat-tolerant | 72 | 38.9 | 185 | 74.9 | Spring | 126 | 68.1 | 120 | 48.6 |
| germplasm | | | | | sowing type | | | | |
| Heat-sensitive | 113 | 61.1 | 62 | 25.1 | Winter | 59 | 31.9 | 127 | 51.4 |
| germplasm | | | | | sowing type | | | | |
| Total number | 185 | 100 | 247 | 100 | Total number | 185 | 100 | 247 | 100 |

The Structure analysis results were verified by principal coordinate analysis (PCoA). PCoA based on the markers related to heat tolerance also divided the screened Pisum sativum germplasms into two genetic subpopulations A and B, which was consistent with the Structure analysis. As shown in FIG. 6B, the subpopulation A in a blue oval (the oval on the left) was roughly separated from the subpopulation B in a red oval (the oval on the right), but there were individual germplasms outside the ovals, wherein an orange diamond represented the subpopulation A of the spring sowing type, an orange triangle represented the subpopulation A of the winter sowing type; a light blue diamond represented the subpopulation B of the spring sowing type, and a light blue triangle represented the subpopulation B of the winter sowing type. The population composition was consistent with that of the Structure analysis. The contribution rate of the first three components of the markers related to heat tolerance in PCoA was 47.47%. The aforementioned results indicated that PCoA well validated the grouping of the genetic subpopulations of Pisum sativum germplasms conducted by Structure analysis.

A phylogenetic tree was constructed by utilizing UPGMA cluster analysis, and thus the analysis results could be displayed more intuitively. An UPGMA dendrogram based on the markers related to heat tolerance also divided the tested Pisum sativum germplasms into two groups of tree branches. As shown in FIG. 6C, the orange tree branch was the subpopulation A, and the light blue tree branch was the subpopulation B. Similar to the PCoA analysis, individual germplasms in one of the two subpopulations were also in the other one of the subpopulations.

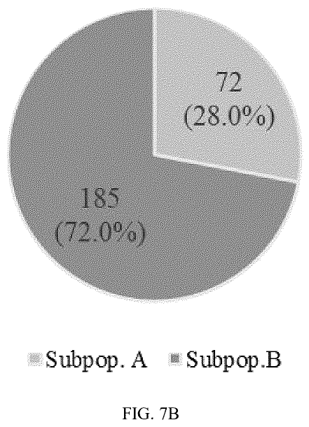

According to different manners, the 432 accessions of Pisum sativum germplasms after the heat tolerance screening could be divided into the spring sowing types (n=246) and the winter sowing type (n=186); and also could be divided into the heat-tolerant germplasm (n=257) and the heat-sensitive germplasm (n=175). 2 subpopulations were obtained through analysis of the population genetic structure of the SNaPshot markers related to heat tolerance, and the genetic composition of heat-tolerant or not could be resolved. As shown in FIGS. 7A-7B, among the 175 accessions of heat-sensitive germplasms, 113 accessions (64.6%) belonged to the subpopulation A, which was nearly 2 times higher than the 62 accessions (35.4%) of the subpopulation B; and among the 257 accessions of heat-tolerant germplasms, only 72 accessions (28.0%) belonged to the subpopulation A, which was less than half of the 185 accessions (72.0%) of the subpopulation B, indicating that most of the heat-sensitive germplasms belonged to the subpopulation A, while most of the heat-tolerant germplasms belonged to the subpopulation B.

### 4. Discussion

In this study, the SNaPshot method was introduced into the identification and evaluation of Pisum sativum germplasms for the first time, and genetic diversity evaluation and population genetic structure analysis were conducted on 432 accessions of Pisum sativum germplasms that had been screened for heat tolerance by utilizing SNaPshot markers related to heat shock proteins or heat shock transcription factors. After analysis of the SNaPshot markers, it was found that the number of the markers significantly affected the total amount of NG and NA, and had a certain impact on the means of MAF, GD and PIC, but had little impact on the mean of He. When the number of markers were increased, the total amount of NG and NA was increased, but the mean of MAF was decreased, the means of GD and PIC were increased, and the proportion of markers with high and medium PIC was increased; and vice versa. From the inside of the marker, the population size has little impact on the total amount of NG and NA, indicating that the marker selection was scientific and the distribution on the chromosome was uniform. The population size was decreased, the mean of MAF was increased, the He did not change much, the means of GD and PIC were decreased, and accordingly the proportion of markers with high and medium PIC was decreased; and vice versa.

The Structure analysis divided the post-screening populations into two genetic subpopulations A and B. The subpopulation A had a total of 185 accessions of germplasms, including 18 accessions (9.7%) of a heat-tolerant and spring-sowing type, 54 accessions (29.2%) of a heat-tolerant and winter-sowing type, 108 accessions (58.4%) of a heat-sensitive and spring-sowing type, and 5 accessions (2.7%) of a heat-sensitive and winter-sowing type, and in the subpopulation A, the heat-sensitive germplasm dominated, and the spring sowing type accounted for the majority; and the subpopulation B had a total of 247 accessions of germplasms, including 82 accessions (33.2%) of the heat-tolerant and spring-sowing type, 103 accessions (41.7%) of the heat-tolerant and winter-sowing type, 38 accessions (15.4%) of the heat-sensitive and spring-sowing type, and 24 accessions (9.7%) of the heat-sensitive and winter-sowing type, and in the subpopulation B, the heat-tolerant germplasm dominated, and the number of the winter-sowing type was slightly higher.

Principal coordinate analysis (PCoA) and UPGMA cluster analysis could verify the results of Structure analysis more intuitively, but there were individual genotypes of different subpopulations within the two subpopulations. The several means of analysis all had verified the experimental results of this study, that was, in subpopulations A and B, there was a certain correlation between the heat tolerance and the type of sowing date, the heat-tolerant germplasms were mostly of the winter sowing type while the heat-sensitive germplasms were mostly of the spring sowing type, which was due to the fact that the spring sowing areas of Pisum sativum were mostly in high latitude areas, such as Liaoning, Inner Mongolia, northern Hebei, Shaanxi, Gansu, Qinghai and the like provinces in northern China, and they were mainly in Europe, North America and the like places when planted abroad, so that high temperature weather above 30° C. rarely occurs in the whole growth period. The local Pisum sativum germplasms were adapted to the cold and cool climate, and the heat-tolerant genes were subjected to less selection pressure and thus did not aggregate, that was, the germplasms did not have the heat-tolerant genes or carry few heat-tolerant genes, so they were very sensitive to high temperature stress. In actual production, the germplasms of the winter sowing type were usually sown in mid-to-late October of the previous year and harvested from April to May of the following year. During the reproductive period of them, high temperature weather above 30° C. were encountered. Therefore, the heat-tolerant genes were retained in the winter sowing type due to the selection pressure, and thus there was a correlation between the heat tolerance of the Pisum sativum germplasms and the type of sowing date. The results of the field heat tolerance screening experiment in this study verified this conclusion. In conclusion, the SNaPshot technology absolutely could be used in the future genetic breeding study of Pisum sativum to accelerate the mining of good Pisum sativum genes.

What is claimed is:

1. A method for conducting heat tolerance screening of Pisum sativum germplasms, comprising:
sowing Pisum sativum germplasms to be screened in three sowing dates: (1) normal sowing (NS); (2) sowing in a first stage of late sowing (LS1); and (3) sowing in a second stage of late sowing (LS2);
utilizing the following equations to calculate rates of average yield loss per plant LR1 and LR2 of each accession of germplasm in the first and second stages of the late sowing:
$$\begin{matrix}LR 1 ( \% ) = [ 1 - ( {P}_{LS 1} / {P}_{NS} ) ] \times 100 \% \\ LR 2 ( \% ) = [ 1 - ( {P}_{LS 2} / {P}_{NS} ) ] \times 100 \%\end{matrix}$$
classifying according to the following heat-tolerant classifying standard of Pisum sativum germplasms:
level 1:0<LR1≤20% and 0<LR2≤20%;
level 2: (0<LR1≤20% and 20%<LR2≤40%) or (20%<LR1≤40% and 0<LR2≤20%);
level 3: 20%<LR1≤40% and 20%<LR2≤40%;
level 4: (20%<LR1<40% and 40%<LR2≤60%) or (40%<LR1≤60% and 20%<LR2≤40%);
level 5: 40%<LR1≤60% and 40%<LR2≤60%;
level 6: (40%<LR1≤60% and 60%<LR2≤80%) or (60%<LR1≤80% and 40%<LR2≤60%);
level 7: 60%<LR1≤80% and 60%<LR2≤80%;
level 8: (60%<LR1≤80% and 80%<LR2≤100%) or (80%<LR1≤100% and 60%<LR2≤80%);
level 9: 80%<LR1≤100% and 80%<LR2≤100%.

2. The method for conducting heat tolerance screening of Pisum sativum germplasms according to claim 1, wherein the sowing in the first stage of the late sowing is later than the normal sowing by 15 days; and the sowing in the second stage of the late sowing is later than the normal sowing by 30 days.

3. A set of SNP markers related to heat tolerance of Pisum sativum developed based on an SNaPshot technology, comprising 20 SNP markers shown in the table below:

| Serial | | |
|---|---|---|
| Number | Marker Name | Sequence |
| 1 | PsCam054965_ | ACCAACCACTGATATCCTAACAGCACTTTCCGAGGACAATCAAAGACTAAGAAGAAAG |
| | 36218_1447 | AA[T/C]CTAATGCTATTATCAGAACTCACTCACATGAAGAATCTCTACAATGACATCATAT |
| | | ATTTC |
| 2 | PsCam040389_ | TTATGCGATTTAGATGCTGAATCTTTAGGAACAATAACAGTGAGAACACCATTTTCAACA |
| | 25143_344 | [T/C]GTGCTTTAATCTGATCCAATTTCACATTCTCAGGCAATTCAATCATCCTTGACAAA |
| | | CCCT |
| 3 | PsCam046167_ | CCTCCAGAAACCGAAAAAACCAACGCCAACGGCAACAACTCTCCTTCAAACTCCGAC |
| | 29682_1689 | GAC[A/G]TCAACGGAGTCGGATCCACCTCCACAGTCCGATCAACCTCTTCCTCCAATTC |
| | | AAAAAACC |
| 4 | PsCam045549_ | ATTTGAGGTAGTGAATGATCATAGTACCAATCATGTAGTTTCATGGAGCAGAGGTGGCA |
| | 29207_370 | C[T/C]AGCTTTGTGATTTGGGATCTACATGCTTTCTCCAATGATCTCCTTCCCAGATACTT |
| | | CAAA |
| 5 | PsCam020968_ | TTTGAATCCGATTATATCATGGAGTTGTAATGGTGCTAGCTTCGTTGTGTGGGACCCTTT[ |
| | 11722_484 | A/G]GAGTTTGCTAGAATCATTTTGCCTCGACATTTCAAACACAACAATTTCTCCAGTTTT |
| | | GTT |
| 6 | PsCam050106_ | TGGAAGCTAGGTTGTTGATTACTGAGAAGAAACATCAACAGATGATGGCTTTTCTTGCA |
| | 32711_1241 | A[A/G]AGCACTCAGTAATCAATCTTTTATTCAGCAATTGGCAAACAACAAAGAGTTGAA |
| | | AGGTGT |
| 7 | PsCam039017_ | CGTAGGAAATCGATTATCCCATATGGGTGTGGAAGGAACAAGTCTCTTTTTGGGTTGAG |
| | 24003_766 | G[T/C]TGGTGCGGTTTGTTGCTTGAACTTGAGACATGGTCCAGTTTTTCTCTTGCGTCGC |
| | | CTGTC |
| 8 | PsCam049061_ | GAATAGGATACCAATTACCTTCTGTTTCTCATTGATGTCAATGTGATCATATTTGGGATC[ |
| | 31705_537 | A/G]TTAGACATTGCAACTTCTCTGTAACTATTGATACAATAGACAAGCTGCTCAATTACT |
| | | GCA |
| 9 | PsCam038211_ | AACAGAATTTTCACTCAATAATTTCTCTGACATAACATGATAAGCAGAAGAAACATCTT |
| | 23256_428 | G[A/C]GCACTTTGTCTAGAATCACCGCAAGCCCTCATTTCATGTAAAGAGCTAACGAATC |
| | | CATTC |
| 10 | PsCam027754_ | ATCCACATGCGCAGACCATATTTTAGCCATTATGTGCCTAATACTCATAGCCAACGGGCG[ |
| | 16321_1079 | A/G]CAACTTCTGGTGTTAGTGTTGCTGCTGCTCCATTAACAGAATCAGAGCGGTGGAAT |
| | | TTTA |
| 11 | PsCam001594_ | AACGTTGATGTTTATCGTCACTCCTTCCCTCGCACTCGCCGCGATGATCTTCTTCTCTCA[ |
| | 1320_450 | A/G]GTAGTTCTTATCGCTCTCTTTTGTTATGTTGATTTGATTCACTAGTATATAATATAAAT |
| | | T |
| 12 | PsCam059391_ | GGGGAAATATGTGTGGAAAGCGTTTAAATTTTGGTGTTTCTAACTCTTATAATATATGTG[ |
| | 39582_1896 | T/C]CGTTTTGTTGTAGTAACAGGTTGAAACATTGGTTTCTCATGTTTGTGATCTCTTTGAT |
| | | AT |
| 13 | PsCam039062_ | TTAATTTGAATTAGATATGGGAAGGGACCTTAAGATATAAAATAATTTGTCCCTATTCAA[ |
| | 24044_1315 | T/G]ACTATTACAATTCAACCACATATAAAATTCAATGTTAGTAGGCGACAGACATGTCTT |
| | | AGA |
| 14 | PsCam013828_ | AAACAATCGTGCAGAGATCATTCATAATGATCAAGGAAACAGAACTACACCTTCTTTTG |
| | 9409_206 | T[T/C]GCTTTTACTGATTCTCAAAGATTGATTGGCGATGCTGCAAAAAATCAGGCTGCTT |
| | | CAAAC |
| 15 | PsCam057184_ | TTTAGTGATTCTGTTATTCAAAATGATTTGAATTTGTGGCCATTCAAAGTCATTTCTGGT[ |
| | 37831_137 | A/G]TCCATGACAAACCCATGATTGTTGTTCAATACAAGGGTGAAGAGAAACACTTTTGT |
| | | GCTG |
| 16 | PsCam036720_ | AGAGAAAGTGAGAGAAAAATAAAGAAAGGAAGGAACGAGAAGGTACTGCGTTAGTTA |
| | 21847_1549 | CGT[T/C]TAATTGTCGAGAAAGTTCCGTGAGAATGAGGATTTTCAGTTTGATTAATGATG |
| | | ATAGAAT |
| 17 | PsCam028698_ | AACAATGAAACTCCTATTTGTAGAACTCCATGAAATAATGGCATTAGTTGTAGGATCATT[ |
| | 16999_180 | T/G]ACCATCATATAAATCTTACACA |
| 18 | PsCam045359_ | GAAGGATAGAGAGAGCATGGCACGAGCCTCTCTAATTGTTCTCGCCATTATTTCAATAG |
| | 29043_269 | G[T/C]AAGTTTTTCGCTGCTTATTATTAGATCTTATGGTTTAATGCCGAAAATGTAACCGA |
| | | TTGA |
| 19 | PsCam035563_ | TTGCTTTCCCCATATTCCACCAAAATCTTCCGTGACCACTAGGTTTTCCTTCATTTTTTC[ |
| | 20741_363 | T/G]GCTATTATTATCCTTTCTTTCTGATTGTACTTCCTGTGCTTCTGCCGCACCAGGATCC |
| | | TC |
| 20 | PsCam042861_ | ATAGGTATTCAACTGCCTGACAAAGCTGGAGAAGTTATTGTGCTTAAAATACTTAGGCA |
| | 26901_2929 | A[A/G]ATATATTTGGAGAAATCGGTGGCATTCAAAACGACAAAGGTGTTATTGTTTTTTC |
| | | CCCAC |

4. The set of SNP markers related to heat tolerance of Pisum sativum developed based on an SNaPshot technology according to claim 3, wherein peripheral amplification primer sequences and single base extension primer sequences for the 20 SNP markers are shown in the table below:
TABLE 4
SNAPshot primer information

| | | Names of | Sequences of | Names of SNP | Sequences of SNP |
|---|---|---|---|---|---|
| | | peripheral | peripheral | single base | single base |
| Serial | Marker | amplification | amplification | extension | extension |
| Number | Name | primers | primers | primers | primers |
| 1 | PsCam054965_ | 1-F | GAACAATCACCTCAA | 1-SNP-F | TTTTTTCAATCAAAGACTAAGA |
| | 36218_ | | ATCCTCCA | | AGAAAGAA |
| | 1447 | 1-R | ACCGATGAATCCAAC | | |
| | | | TCAACAAG | | |
| 2 | PsCam040389_ | 2-F | TAACCGCCAAGATCA | 2-SNP-F | TTTTTTTTTTTTTTTTTTTTTTTA |
| | 25143_ | | AGGGA | | GTGAGAACACCATTTTCAACA |
| | 344 | 2-R | GCTGAGAGAGGGAC | | |
| | | | TGGAAA | | |
| 3 | PsCam046167_ | 3-F | TCCTCACCGAAATCA | 3-SNP-F | TTTTTTTTTTTTTTTTTCCTTCA |
| | 29682_ | | AACGC | | AACTCCGACGAC |
| | 1689 | 3-R | AAAGACACCAACTCA | | |
| | | | TCGCACT | | |
| 4 | PsCam045549_ | 4-F | AACAAGCATTGATTC | 4-SNP-F | TTTTTTTTTTTTTTTTTTTTTTAT |
| | 29207_ | | TACCACCC | | GGAGCAGAGGTGGCAC |
| | 370 | 4-R | ACAAACAACAAAAG | | |
| | | | CAAGCCG | | |
| 5 | PsCam020968_ | 5-F | GCCTATGCCTATGGA | 5-SNP-F | TTTTTTTTTTTTTTTTTTTTTTTT |
| | 11722_ | | GTGTTTG | | TTTCGTTGTGTGGGACCCTTT |
| | 484 | 5-R | TTACATTTAGAGGGT | | |
| | | | GACAAAGCAA | | |
| 6 | PsCam050106_ | 6-F | CAACGAGAAAGTGG | 6-SNP-F | TTTTTTTTTTTTTTTTTTTTTTTT |
| | 32711_ | | TGGTGC | | TTTTTTATGATGGCTTTTCTTGC |
| | 1241 | 6-R | AGCCTCCTCTTTCGC | | AA |
| | | | TTCAT | | |
| 7 | PsCam039017_ | 7-F | TCCTTCTTTAATCTCC | 7-SNP-R | TTTTTTTTTTTTTTTTTTTTTTTT |
| | 24003_ | | CACGG | | TTTTTTTTTTTAAGCAACAAAC |
| | 766 | 7-R | ATTGAAACCAACTCA | | CGCACCA |
| | | | CTTCCCTT | | |
| 8 | PsCam049061_ | 8-F | AAGCCAGTTCTCAGC | 8-SNP-F | TTTTTTTTTTTTTTTTTTTTTTTT |
| | 31705_ | | TTCTCC | | TTTTTTTCAATGTGATCATATTT |
| | 537 | 8-R | TCGAGGAGCGCTACA | | GGGATC |
| | | | AAGAA | | |
| 9 | PsCam038211_ | 9-F | TCCAACCAAACCACT | 9-SNP-R | TTTTTTTTTTTTTTTTTTTTTTTT |
| | 23256_ | | AGCAGATC | | TTTTTTTTTTTTCGGTGATTCTA |
| | 428 | 9-R | AGGCCTCAAGGTCGA | | GACAAAGTGC |
| | | | GAACAG | | |
| 10 | PsCam027754_ | 10-F | TGATCAGGGCTTTAG | 10-SNP-R | TTTTTTTTTTTTTTTTTTTTTTTT |
| | 16321_ | | AAAGGTTG | | TTTGCAACACTAACACCAGAA |
| | 1079 | 10-R | CACTCTTCTTGCTGC | | GTTG |
| | | | CTCTGAC | | |
| 11 | PsCam001594_ | 11-F | TCAAGCTCTTTTCTTC | 11-SNP-R | TTTTTTTTTTTTTTTTTTTTTTTT |
| | 1320_ | | GCCC | | TTTTTTTTTTTTTACAAAAGAG |
| | 450 | 11-R | CTGTCTCTCTCGCATC | | AGCGATAAGAACTAC |
| | | | CCATC | | |
| 12 | PsCam059391_ | 12-F | TGGAAACTTGGTGTG | 12-SNP-R | TTTTTTTCAACCTGTTACTACA |
| | 39582_ | | ACAGTAAATC | | ACAAAACG |
| | 1896 | 12-R | TCAACTATCAGAGAT | | |
| | | | TCTTGTCCAA | | |
| 13 | PsCam039062_ | 13-F | AGCCATTTGTTTTGTT | 13-SNP-F | TTTTTTTTTTTATAAAATAATTT |
| | 24044_ | | TTCTTGAG | | GTCCCTATTCAA |
| | 1315 | 13-R | TCATTGCAAAATTCC | | |
| | | | CATCTACTT | | |
| 14 | PsCam013828_ | 14-F | TGTGTAGCAGTTTGG | 14-SNP-F | TTTTTTAAACAGAACTACACCT |
| | 9409_ | | CAGGG | | TCTTTTGT |
| | 206 | 14-R | AGGTTTGTCATCTTTG | | |
| | | | CCAGC | | |
| 15 | PsCam057184_ | 15-F | GCTTCTAACCCAACC | 15-SNP-F | TTTTTTTTTTTTTTTTTTTCCAT |
| | 37831_ | | AATACAGTC | | TCAAAGTCATTTCTGGT |
| | 137 | 15-R | ACTTCACGCATCTTTC | | |
| | | | TGAGGAC | | |
| 16 | PsCam036720_ | 16-F | ATAGGGGGTGTTATT | 16-SNP-F | TTTTTTTTTTTTTTTTTTTTTTA |
| | 21847_ | | GTGGTGAT | | GAAGGTACTGCGTTAGTTACGT |
| | 1549 | 16-R | GATTGCGAAGAGCCG | | |
| | | | TGAA | | |
| 17 | PsCam028698_ | 17-F | AACTCGAGAAATTGT | 17-SNP-F | TTTTTTTTTTTTTTTTTTTTTTTT |
| | 16999_ | | TGTGCCTA | | TTTTGGCATTAGTTGTAGGATC |
| | 180 | 17-R | TGAGATTGTGCTACTA | | ATT |
| | | | TGGAGCAA | | |
| 18 | PsCam045359_ | 18-F | TCACCGTCTTCACCA | 18-SNP-F | TTTTTTTTTTTTTTTTTTTTTTTT |
| | 29043_ | | TTACACC | | TTTTTTTTTCTCGCCATTATTTC |
| | 269 | 18-R | ACAGTTGAACAAAAC | | AATAGG |
| | | | CTGATCCA | | |
| 19 | PsCam035563_ | 19-F | GGCATCACAACCAGA | 19-SNP-F | TTTTTTTTTTTTTTTTTTTTTTTT |
| | 20741_ | | AGAAATAGT | | TTTTTTTTATCAGAAAGAAAGG |
| | 363 | 19-R | GCCTGTAGGAATCAC | | ATAATAATAGC |
| | | | CACTGC | | |
| 20 | PsCam042861_ | 20-F | GGTATTCAACTGCCT | 20-SNP-F | TTTTTTTTTTTTTTTTTTTTTTTT |
| | 26901_ | | GACAAAGC | | TTTTTTTTTTTTTTTTGTGCTTA |
| | 2929 | 20-R | CTACGACATGGTGGA | | AAATACTTAGGCAA |
| | | | TGACTCTTC | | |

5. Use of the SNP markers related to heat tolerance of Pisum sativum according to claim 3 in analysis of genetic diversity and population genetic structure of heat-tolerant and heat-sensitive Pisum sativum germplasms.

6. Use of the SNP markers related to heat tolerance of Pisum sativum according to claim 4 in analysis of genetic diversity and population genetic structure of heat-tolerant and heat-sensitive Pisum sativum germplasms.

7. Use of the SNP markers related to heat tolerance of Pisum sativum according to claim 3 in study of heat-tolerant genetic mechanism and heat-tolerant breeding of Pisum sativum.

8. Use of the SNP markers related to heat tolerance of Pisum sativum according to claim 4 in study of heat-tolerant genetic mechanism and heat-tolerant breeding of Pisum sativum.

9. A method for analyzing genetic diversity of heat-tolerant and heat-sensitive Pisum sativum germplasms by employing the SNP markers related to heat tolerance of Pisum sativum according to claim 4, comprising:
1) SNaPshot PCR reaction
First, heat tolerance screening of rice germplasms is performed, and establishing a genetic population comprising heat-tolerant and heat-sensitive Pisum sativum germplasms; and
then conducting peripheral amplification by using DNAs of the population of Pisum sativum germplasms to be tested as PCR templates with each locus being subjected to single amplification, purifying PCR products and then conducting SNaPshot PCR of them by employing single base extension primers, and detecting reaction products of the SNaPshot PCR by capillary electrophoresis via an ABI 3730XL DNA analyzer; and
2) genetic diversity analysis
conducting data analysis of SNP loci by utilizing Gene mapper 4.1, wherein each sample is genotyped according to peaks corresponding to the SNP loci, and the resultant analysis results are a file of an Excel format and a peak map of a PDF format, and calculating genetic diversity parameters of two groups of SNP markers by utilizing PowerMarker 3.25.

10. The method for analyzing genetic diversity of heat-tolerant and heat-sensitive Pisum sativum germplasms according to claim 7, wherein the genetic diversity parameters in the step 2) comprises a number of genotypes, a major allele frequency, a number of alleles, gene diversity, expected heterozygosity and polymorphic information content.

11. A method for analyzing genetic diversity of heat-tolerant and heat-sensitive Pisum sativum germplasms, comprising, on the basis of step 2) of claim 9, firstly calculating genetic composition of the Pisum sativum germplasms by utilizing Structure 2.3.4, and determining an optimal grouping number of genetic subpopulations of them; secondly verifying the analysis result of Structure by utilizing principal coordinate analysis PCoA; and finally constructing a phylogenetic tree by utilizing UPGMA cluster analysis to display the analysis result intuitively.

12. A method for analyzing genetic diversity of heat-tolerant and heat-sensitive Pisum sativum germplasms, comprising, on the basis of step 2) of claim 10, firstly calculating genetic composition of the Pisum sativum germplasms by utilizing Structure 2.3.4, and determining an optimal grouping number of genetic subpopulations of them; secondly verifying the analysis result of Structure by utilizing principal coordinate analysis PCoA; and finally constructing a phylogenetic tree by utilizing UPGMA cluster analysis to display the analysis result intuitively.


SEQUENCE LISTING (40 sequences)

SEQ ID NO: 1 - DNA, 23 bp, synthetic construct
  source 1..23: mol_type = other DNA
gaacaatcacctcaaatcctcca

SEQ ID NO: 2 - DNA, 23 bp, synthetic construct
  source 1..23: mol_type = other DNA
accgatgaatccaactcaacaag

SEQ ID NO: 3 - DNA, 20 bp, synthetic construct
  source 1..20: mol_type = other DNA
taaccgccaagatcaaggga

SEQ ID NO: 4 - DNA, 20 bp, synthetic construct
  source 1..20: mol_type = other DNA
gctgagagagggactggaaa

SEQ ID NO: 5 - DNA, 20 bp, synthetic construct
  source 1..20: mol_type = other DNA
tcctcaccgaaatcaaacgc

SEQ ID NO: 6 - DNA, 22 bp, synthetic construct
  source 1..22: mol_type = other DNA
aaagacaccaactcatcgcact

SEQ ID NO: 7 - DNA, 23 bp, synthetic construct
  source 1..23: mol_type = other DNA
aacaagcattgattctaccaccc

SEQ ID NO: 8 - DNA, 21 bp, synthetic construct
  source 1..21: mol_type = other DNA
acaaacaacaaaagcaagccg

SEQ ID NO: 9 - DNA, 22 bp, synthetic construct
  source 1..22: mol_type = other DNA
gcctatgcctatggagtgtttg

SEQ ID NO: 10 - DNA, 25 bp, synthetic construct
  source 1..25: mol_type = other DNA
ttacatttagagggtgacaaagcaa

SEQ ID NO: 11 - DNA, 20 bp, synthetic construct
  source 1..20: mol_type = other DNA
caacgagaaagtggtggtgc

SEQ ID NO: 12 - DNA, 20 bp, synthetic construct
  source 1..20: mol_type = other DNA
agcctcctctttcgcttcat

SEQ ID NO: 13 - DNA, 21 bp, synthetic construct
  source 1..21: mol_type = other DNA
tccttctttaatctcccacgg

SEQ ID NO: 14 - DNA, 23 bp, synthetic construct
  source 1..23: mol_type = other DNA
attgaaaccaactcacttccctt

SEQ ID NO: 15 - DNA, 21 bp, synthetic construct
  source 1..21: mol_type = other DNA
aagccagttctcagcttctcc

SEQ ID NO: 16 - DNA, 20 bp, synthetic construct
  source 1..20: mol_type = other DNA
tcgaggagcgctacaaagaa

SEQ ID NO: 17 - DNA, 23 bp, synthetic construct
  source 1..23: mol_type = other DNA
tccaaccaaaccactagcagatc

SEQ ID NO: 18 - DNA, 21 bp, synthetic construct
  source 1..21: mol_type = other DNA
aggcctcaaggtcgagaacag

SEQ ID NO: 19 - DNA, 23 bp, synthetic construct
  source 1..23: mol_type = other DNA
tgatcagggctttagaaaggttg

SEQ ID NO: 20 - DNA, 22 bp, synthetic construct
  source 1..22: mol_type = other DNA
cactcttcttgctgcctctgac

SEQ ID NO: 21 - DNA, 20 bp, synthetic construct
  source 1..20: mol_type = other DNA
tcaagctcttttcttcgccc

SEQ ID NO: 22 - DNA, 21 bp, synthetic construct
  source 1..21: mol_type = other DNA
ctgtctctctcgcatcccatc

SEQ ID NO: 23 - DNA, 25 bp, synthetic construct
  source 1..25: mol_type = other DNA
tggaaacttggtgtgacagtaaatc

SEQ ID NO: 24 - DNA, 25 bp, synthetic construct
  source 1..25: mol_type = other DNA
tcaactatcagagattcttgtccaa

SEQ ID NO: 25 - DNA, 24 bp, synthetic construct
  source 1..24: mol_type = other DNA
agccatttgttttgttttcttgag

SEQ ID NO: 26 - DNA, 24 bp, synthetic construct
  source 1..24: mol_type = other DNA
tcattgcaaaattcccatctactt

SEQ ID NO: 27 - DNA, 20 bp, synthetic construct
  source 1..20: mol_type = other DNA
tgtgtagcagtttggcaggg

SEQ ID NO: 28 - DNA, 21 bp, synthetic construct
  source 1..21: mol_type = other DNA
aggtttgtcatctttgccagc

SEQ ID NO: 29 - DNA, 24 bp, synthetic construct
  source 1..24: mol_type = other DNA
gcttctaacccaaccaatacagtc

SEQ ID NO: 30 - DNA, 23 bp, synthetic construct
  source 1..23: mol_type = other DNA
acttcacgcatctttctgaggac

SEQ ID NO: 31 - DNA, 23 bp, synthetic construct
  source 1..23: mol_type = other DNA
atagggggtgttattgtggtgat

SEQ ID NO: 32 - DNA, 19 bp, synthetic construct
  source 1..19: mol_type = other DNA
gattgcgaagagccgtgaa

SEQ ID NO: 33 - DNA, 23 bp, synthetic construct
  source 1..23: mol_type = other DNA
aactcgagaaattgttgtgccta

SEQ ID NO: 34 - DNA, 24 bp, synthetic construct
  source 1..24: mol_type = other DNA
tgagattgtgctactatggagcaa

SEQ ID NO: 35 - DNA, 22 bp, synthetic construct
  source 1..22: mol_type = other DNA
tcaccgtcttcaccattacacc

SEQ ID NO: 36 - DNA, 23 bp, synthetic construct
  source 1..23: mol_type = other DNA
acagttgaacaaaacctgatcca

SEQ ID NO: 37 - DNA, 24 bp, synthetic construct
  source 1..24: mol_type = other DNA
ggcatcacaaccagaagaaatagt

SEQ ID NO: 38 - DNA, 21 bp, synthetic construct
  source 1..21: mol_type = other DNA
gcctgtaggaatcaccactgc

SEQ ID NO: 39 - DNA, 23 bp, synthetic construct
  source 1..23: mol_type = other DNA
ggtattcaactgcctgacaaagc

SEQ ID NO: 40 - DNA, 24 bp, synthetic construct
  source 1..24: mol_type = other DNA
ctacgacatggtggatgactcttc